(19) United States
(12) Patent Application Publication
(10) Pub. No.: US 2026/0327714 A1
(43) Pub. Date: Oct. 22, 2026

(54) Assays for Detection of Isocitrate Dehydrogenase 1 R132H (IDH1.R132H) Mutant Cancers

(71) Applicant: The General Hospital Corporation, Boston, MA (US)

(72) Inventors: Leonora Balaj, Lincoln, MA (US); Bob S. Carter, Belmont, MA (US); Syeda M. Batool, Boston, MA (US)

(21) Appl. No.: 19/578,534

(22) Filed: Mar. 25, 2026

Related U.S. Application Data
(60) Provisional application No. 63/777,083, filed on Mar. 25, 2025.

(51) Int. Cl. C12Q 1/6886

(52) U.S. Cl. CPC C12Q 1/6886; C12Q 2600/156

(57) ABSTRACT

Described herein are sensitive assays for detection of IDH1.R132H-mutant cancers, e.g., gliomas. The assays generally use droplet digital PCR (ddPCR) to detect the IDH1.R132H mutation in plasma-derived EV mRNA, or in RNA from tumor cells or tissue.

### CLAIM OF PRIORITY

This application claims the benefit of U.S. Provisional Application Ser. No. 63/777,083, filed on Mar. 25, 2025. The entire contents of the foregoing are incorporated herein by reference.

### FEDERALLY SPONSORED RESEARCH OR DEVELOPMENT

This invention was made with Government support under Grant Nos. CA230697, CA239078, and CA237500 awarded by the National Institutes of Health. The Government has certain rights in the invention.

### SEQUENCE LISTING

This application contains a Sequence Listing that has been submitted electronically as an XML file named “29539-0822001_SL_ST26.XML.” The XML file, created on May 21, 2026, is 34,978 bytes in size. The material in the XML file is hereby incorporated by reference in its entirety.

### TECHNICAL FIELD

Described herein are assays, e.g., blood-based assays, for detection of Isocitrate Dehydrogenase 1 R132H (IDH1.R132H) mutant cancers, e.g., gliomas. The assays generally use droplet digital PCR (ddPCR) to detect the IDH1.R132H mutation in plasma derived EV mRNA.

### BACKGROUND

Glioma is the most common primary central nervous system (CNS) neoplasm in adults with an overall incidence of 6 cases per 100,000 people in the US every yearE. The 2021 WHO classification of brain tumors provides a new framework for comprehensive characterization of gliomas based on histology and independently by hallmark molecular alterations. Importantly, IDH1 mutant alteration represents a key diagnostic biomarker, prevalent in >80% of WHO grade II/III gliomas², >70% of Secondary Glioblastoma (GBM)³, and <10% of Primary GBM⁴ (now classified as Grade 4, Astrocytoma). This point mutation is found in codon 132, characterized by G>A transition (CGT>CAT) at nucleotide position 395, resulting in the substitution of arginine for histidine³. IDH1 mutant gliomas represent a distinctive subtype with important clinical implications, most importantly improved prognosis and survival outcomes5.

Currently, neuroimaging followed by tissue biopsy testing (surgical biopsy and/or resection) is the current gold standard modality for diagnosing IDH1 mutant gliomas⁶. A range of testing platforms are used with varying sensitivity and depth of coverage and include immunohistochemistry (IHC), DNA based PCR, and Next Generation Sequencing (NGS)⁷. The turnaround time for diagnosis from the point of intraoperative sampling is around 2-3 weeks. Furthermore, in addition to being invasive, surgical tissue biopsy relies on spatial sampling (lesion edge, core) and availability of sufficient and adequate tissue⁸. Furthermore, tissue biopsy can not be readily repeated in longitudinal settings for disease monitoring. This serves as a major limitation in tracking the dynamic, evolving molecular profile secondary to treatment, surgical intervention, and/or natural disease progression. Furthermore, a recent study has also reported a detrimental effect on overall survival of tumor tissue biopsy over “upfront resection” in high grade gliomas⁹.

### SUMMARY

Described herein is a blood-based droplet digital PCR (ddPCR) assay that can detect IDH1.R132H mutation in plasma derived EV mRNA. In some embodiments, the assay has an overall sensitivity of 75.0% (95% CI: 64.1%-84.0%), specificity 88.7% (95% CI: 77.0%-95.7%), and area under the curve (AUC) of 0.84 (p<0.0001). In addition to baseline diagnosis, there is also a correlation between IDH1.R132H mutant allele frequency (MAF) (tumor tissue, plasma) and key clinical variables: (i) overall survival, (ii) tumor shedding rate, (ii) treatment outcomes. The described assays can be utilized, e.g., for blood-based diagnosis, prognostication, treatment selection, and longitudinal monitoring.

Provided herein are methods for detecting a mutation at position R132 in Isocitrate Dehydrogenase 1 gene (IDH1) in a sample comprising a biofluid from a subject. The methods comprise providing a sample comprising ribonucleic acids isolated from extracellular vesicles (EV RNA) extracted from the sample, reverse transcribing the EV RNA to generate EV cDNA; performing a preamplification reaction by contacting the EV cDNA with a pair of amplification primers comprising a forward primer and a reverse primer, in the presence of a wild-type blocking oligonucleotide; performing a further amplification reaction by contacting the preamplified sample with the pair of amplification primers comprising a forward primer and a reverse primer, and a pair of detection primers (also referred to herein as detection probes) comprising a mutant primer and a wild-type primer, wherein the mutant primer comprises a first detectable label and the wild-type primer comprises a second detectable label, under conditions sufficient for amplifying the IDH1 sequence, and detecting a signal from the first detectable label and/or the second detectable label, wherein presence of the signal from the first detectable label indicates presence of a mutant IDH1 sequence in the sample and/or wherein presence of the signal from the second detectable label indicates presence of a wild-type IDH1 promoter sequence in the sample. In some embodiments, the sample comprises serum, saliva, urine, or cerebrospinal fluid.

Also provided herein are methods for detecting a mutation at position R132 in Isocitrate Dehydrogenase 1 gene (IDH1) in a sample comprising tumor cells or tissue from a subject. The methods comprise: providing a sample comprising ribonucleic acids isolated from the sample, reverse transcribing the RNA to generate cDNA; performing a preamplification reaction by contacting the cDNA with a pair of amplification primers comprising a forward primer and a reverse primer, in the presence of a wild-type blocking oligonucleotide; performing a further amplification reaction by contacting the preamplified sample with the pair of amplification primers comprising a forward primer and a reverse primer, and a pair of detection primers comprising a mutant detection primer and a wild-type detection primer, wherein the mutant primer comprises a first detectable label and the wild-type primer comprises a second detectable label, under conditions sufficient for amplifying the IDH1 sequence, and detecting a signal from the first detectable label and/or the second detectable label, wherein presence of the signal from the first detectable label indicates presence of a mutant IDH1 sequence in the sample and/or wherein presence of the signal from the second detectable label indicates presence of a wild-type IDH1 promoter sequence in the sample.

In some embodiments, the mutation is R132H, and: the forward primer comprises SEQ ID NO: 1 and the reverse primer comprises SEQ ID NO: 2, the mutant detection primer comprises SEQ ID NO: 4 or 24, the wild-type detection primer comprises SEQ ID NO: 5 or 25; and/or the wild-type blocking oligonucleotide comprises SEQ ID NO:8. Alternatively, the reverse primer can comprise SEQ ID NO:18 or 19.

In some embodiments, the forward primer comprises a nucleotide sequence that is at least 80% identical, at least 85%, at least 90%, at least 91%, at least 92%, at least 93%, at least 94%, at least 95%, at least 96%, at least 97%, at least 98%, or at least 99% identical to SEQ ID NO: 1. In some embodiments, the forward primer comprises SEQ ID NO: 1. In some embodiments, the forward primer is SEQ ID NO: 1.

In some embodiments, the reverse primer comprises a nucleotide sequence that is at least 80% identical, at least 85%, at least 90%, at least 91%, at least 92%, at least 93%, at least 94%, at least 95%, at least 96%, at least 97%, at least 98%, or at least 99% identical to SEQ ID NO: 2, 18, or 19. In some embodiments, the reverse primer comprises SEQ ID NO: 2, 18, or 19. In some embodiments, the reverse primer is SEQ ID NO: 2.

In some embodiments, the mutant detection primer comprises a nucleotide sequence that is at least 80% identical, at least 85%, at least 90%, at least 91%, at least 92%, at least 93%, at least 94%, at least 95%, at least 96%, at least 97%, at least 98%, or at least 99% identical to SEQ ID NO: 4 or 24. In some embodiments, the mutant detection primer comprises SEQ ID NO: 4 or 24. In some embodiments, the mutant detection primer is SEQ ID NO: 4.

In some embodiments, the wild-type detection primer comprises a nucleotide sequence that is at least 80% identical, at least 85%, at least 90%, at least 91%, at least 92%, at least 93%, at least 94%, at least 95%, at least 96%, at least 97%, at least 98%, or at least 99% identical to SEQ ID NO: 5 or 25. In some embodiments, the wild-type detection primer comprises SEQ ID NO: 5 or 25. In some embodiments, the wild-type detection primer is SEQ ID NO: 5.

In some embodiments, the wild-type blocking oligonucleotide comprises a nucleotide sequence that is at least 80% identical, at least 85%, at least 90%, at least 91%, at least 92%, at least 93%, at least 94%, at least 95%, at least 96%, at least 97%, at least 98%, or at least 99% identical to SEQ ID NO: 8. In some embodiments, the forward primer comprises SEQ ID NO: 8. In some embodiments, the forward primer is SEQ ID NO: 8.

In some embodiments, the further amplification reaction is droplet digital PCR (ddPCR).

In some embodiments, the wild type blocking oligonucleotide comprises at least one locked nucleic acid (LNA) modification.

In some embodiments, the detection primers each comprise a fluorophore and a quencher.

In some embodiments, the mutant detection primers comprise a first fluorophore and a first quencher, and the second detection primers comprise a second fluorophore and a second quencher.

In some embodiments, the first fluorophore and the second fluorophore are different fluorophores, and/or wherein the first quencher and the second quencher are the same quencher.

In some embodiments, the first fluorophore and the second fluorophore are selected from the group consisting of FAM, HEX, Cy3, Cy5, and Texas Red, and/or wherein the first quencher and the second quencher are selected from the group consisting of Iowa Black FQ, Iowa Black RQ, ZEN Quencher, and TAMRA.

In some embodiments, the subject is a human patient having or suspected of having a cancer.

In some embodiments, the cancer is selected from glioma, myeloid malignancies, chondrosarcoma, cholangiocarcinoma, astrocytoma, secondary glioblastoma (diffuse astrocytoma), ependymoma, primary glioblastoma, cartilaginous and bone tumors, intrahepatic cholangiocarcinoma, acute myeloid leukemia.

In some embodiments, the brain cancer is a glioma, optionally wherein the glioma is an astrocytoma, an ependymoma, or an oligodendroglioma, or the myeloid malignancy is acute myeloid leukemia (AML) and myelodysplastic syndromes (MDS).

In some embodiments, the methods further comprise administering a cancer therapy to the subject when the subject has been identified as having an IDH1 mutation. In some embodiments, the cancer therapy is selected from the group consisting of a chemotherapy (optionally an IDH-targeted therapy), a radiation therapy, a surgical therapy, and an immunotherapy.

Additionally, provided herein are methods of evaluating reoccurrence of a cancer in a subject. The methods comprise: detecting the IDH1 sequence in a sample from the subject according to a method described herein, determining a level of IDH1 mutant sequences in the sample and comparing the level to a reference level; determining that the subject has reoccurrence of the cancer, when the subject is identified as having a level of IDH1 mutant sequences in the sample above the reference level, and optionally administering a cancer therapy to the subject identified as having a reoccurrence of the cancer.

Further, provided herein are methods of evaluating effectiveness of a cancer therapy. The methods comprise: in a sample from the subject who has been administered the cancer therapy, detecting the IDH1 sequence according to a method as described herein, determining a level of IDH1 mutant sequences in the sample and comparing the level to a reference level, optionally wherein the reference level is a level determined in the subject at an earlier time point, optionally before the administration of the therapy; determining that the cancer therapy has been effective when the level of mutant IDH1 sequences in the sample is below the reference level, and optionally administering the cancer therapy identified as effective to the subject and/or administering another cancer therapy to the subject.

Also provided herein are kits for amplifying a region of a human IDH1 gene. The kits can comprise one, two, three, or all four of: a forward primer that comprises SEQ ID NO: 1; a reverse primer that comprises SEQ ID NO: 2; a mutant detection primer that comprises SEQ ID NO: 4 or 24; a wild-type detection primer that comprises SEQ ID NO: 5 or 25; and/or a wild-type blocking oligonucleotide that comprises SEQ ID NO:8, and reagents (and optionally instructions) for performing a method as described herein. Alternatively, the reverse primer can comprise SEQ ID NO:18 or 19.

In some embodiments, the forward primer comprises a nucleotide sequence that is at least 80% identical, at least 85%, at least 90%, at least 91%, at least 92%, at least 93%, at least 94%, at least 95%, at least 96%, at least 97%, at least 98%, or at least 99% identical to SEQ ID NO: 1. In some embodiments, the forward primer comprises SEQ ID NO: 1. In some embodiments, the forward primer is SEQ ID NO: 1.

In some embodiments, the reverse primer comprises a nucleotide sequence that is at least 80% identical, at least 85%, at least 90%, at least 91%, at least 92%, at least 93%, at least 94%, at least 95%, at least 96%, at least 97%, at least 98%, or at least 99% identical to SEQ ID NO: 2, 18, or 19. In some embodiments, the reverse primer comprises SEQ ID NO: 2, 18, or 19. In some embodiments, the reverse primer is SEQ ID NO: 2.

In some embodiments, the mutant detection primer comprises a nucleotide sequence that is at least 80% identical, at least 85%, at least 90%, at least 91%, at least 92%, at least 93%, at least 94%, at least 95%, at least 96%, at least 97%, at least 98%, or at least 99% identical to SEQ ID NO: 4 or 24. In some embodiments, the mutant detection primer comprises SEQ ID NO: 4 or 24. In some embodiments, the mutant detection primer is SEQ ID NO: 4.

In some embodiments, the wild-type detection primer comprises a nucleotide sequence that is at least 80% identical, at least 85%, at least 90%, at least 91%, at least 92%, at least 93%, at least 94%, at least 95%, at least 96%, at least 97%, at least 98%, or at least 99% identical to SEQ ID NO: 5 or 25. In some embodiments, the wild-type detection primer comprises SEQ ID NO: 5 or 25. In some embodiments, the wild-type detection primer is SEQ ID NO: 5.

In some embodiments, the wild-type blocking oligonucleotide comprises a nucleotide sequence that is at least 80% identical, at least 85%, at least 90%, at least 91%, at least 92%, at least 93%, at least 94%, at least 95%, at least 96%, at least 97%, at least 98%, or at least 99% identical to SEQ ID NO: 8. In some embodiments, the forward primer comprises SEQ ID NO: 8. In some embodiments, the forward primer is SEQ ID NO: 8.

In some embodiments, the reagents are for performing EV isolation from serum and/or droplet digital PCR (ddPCR).

In some embodiments, the wild type blocking oligonucleotide comprises at least one locked nucleic acid (LNA) modification.

In some embodiments, the detection primers each comprise a fluorophore and a quencher, optionally wherein the mutant detection primers comprise a first fluorophore and a first quencher, and the second detection primers comprise a second fluorophore and a second quencher.

In some embodiments, the first fluorophore and the second fluorophore are different fluorophores, and/or wherein the first quencher and the second quencher are the same quencher, optionally wherein the first fluorophore and the second fluorophore are selected from the group consisting of FAM, HEX, Cy3, Cy5, and Texas Red, and/or wherein the first quencher and the second quencher are selected from the group consisting of Iowa Black FQ, Iowa Black RQ, ZEN Quencher, and TAMRA.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-H: RNA-based approach enables higher sensitivity of mutation analysis compared to DNA-based detection. Multi-phase testing of IDH1.R132H, mutant allele frequency (MAF, %) enrichment in RNA versus DNA: (a) Stacked Graph depicting IDH1.R132H MAF % (average of three replicates) in tumor tissue (n=1) derived RNA/DNA in a range equal ddPCR inputs (30.0-0.30) ng, (b) Violin plot comparing IDH1.R132H MAF, % RNA vs DNA Fold-Change (FC=RNA, MAF %/DNA, MAF %) across three glioma subtypes (Astrocytoma, Grade 2-3; n=12, Oligodendroglioma, Grade 2-3; n=7, Astrocytoma, Grade 4; n=4). Each data point represents an individual patient (average of three technical replicates). Average FC was compared across the groups using one way ANOVA (alpha 0.05, Tukey's multiple comparisons). Results of mean difference across multiple comparisons: Astrocytoma, Grade 2, 3 vs. Oligodendroglioma, Grade 2, 3 (−5.718, p=0.0025), Astrocytoma, Grade 2,-3 vs Grade 4 (−2.828, p=0.2749), and Oligodendroglioma vs Astrocytoma, Grade 4 (2.889, p=0.3159) (c) Bar-graph depicting the spatial variation in IDH1.R132H MAF, % RNA vs DNA Fold-Change (mean±SEM) in spatial (multi-region; core, edge) samples analyzed from astrocytoma (n=1) and oligodendroglioma (n 2), and (d) Sensitivity of IDH1.R132H (left, mean±SEM; 4 replicates) and IDH1 wild-type (right, mean±SEM; 4 replicates) detection in a cohort (n=5) of 2 ml plasma derived EV RNA (EXOLUTION™ EV RNA isolation kit) versus EV RNA+EV DNA+cfDNA (EXOLUTIONPLUS™ EV RNA/DNA isolation kit). Results of IDH1.R132H copy number (left) showed no detection in P1-P3 and H1-H2 using EXOLUTIONPLUS™. For P1-P3 the bars denote mean±SEM (P1; 3.081, 95% CI: 1.072-5.090, P2: 322.849, 95% CI: 203.57-422.341, P3: 18.091, 95% CI: −15.345-51.526). Multiple unpaired t tests were performed to compare the difference in IDH1 wild-type copy number (right) across the individual patients (P1-P3) and controls (H1-H2). Across all individual patients and controls, p value<0.000001 when comparing mean differences (mean±SEM): (P1; 5876±44.43, P2: 2619±109.6, P3: 1064±49.41, H1: 1382±60.72, H2: 1018±40.54). (e) Violin plot depicting variation of IDH1.R132H MAF % across three different optimization conditions (4 technical replicates per condition) of preamplification PCR. Average MAF % was compared across the groups using one way ANOVA (alpha 0.05, Tukey's multiple comparisons). Results of mean difference across multiple comparisons: Primers vs. (Primers+Mutant Probe) (−17.46, p value<0.0001), Primers vs (Primers+Mutant, Wild-type Probe) (−2.191, p value 0.0145), and (Primers+Mutant Probe) vs (Primers+Mutant, Wild-type Probe) (15.27, p value<0.0001). (f) Preamplification of IDH1 wild-type tumor tissue and healthy plasma with (+, 3 technical replicates) and without (−, 3 technical replicates) the presence of an optimized wild-type blocker to compare the false positive signal. Results plotted as a bar graph (mean±SEM). Average MAF % was compared across the groups using one way ANOVA (alpha 0.05, Sidak's multiple comparisons). Results of mean difference across multiple comparisons: Wild-type tumor tissue, no blocker vs. blocker (0.1381, p value<0.0001), Healthy Plasma, no blocker vs blocker (0.0522, p value<0.0001). (g) Assessment of optimized Droplet Digital PCR (ddPCR) assay linearity and determination of Limit of Detection (LOD) and Limit of Blank (LOB). At each input concentration, n=3 technical replicates were run at the ddPCR step. Each data point on the line graph denotes Mean±SEM, 3 technical replicates. (h) 2-D ddPCR plots of highest and lowest IDH1.R132H MAF, % detected.

FIGS. 2A-J: Enhanced performance of the mt-IDH1_{dx} assay in tumor tissue across the glioma subtypes. (a) Schematic comparing the modalities routinely applied in clinical settings for determination of IDH1.R132H status versus the one-step laboratory developed Droplet Digital PCR platform, referred to as mt-IDH1_{dx} ddPCR assay, for rapid, tumor tissue diagnostics. (b) mt-IDH1_{dx} assay performance evaluated in tumor tissue cohort (N=48) in comparison to clinical pathology testing across different histological subtypes: (c) Astrocytoma, Grade 2, 3 (n=25, biological replicates), (d) Astrocytoma, Grade 4 (n=12, biological replicates), and (e) Oligodendroglioma, Grade 2, 3 (n=11, biological replicates). (f-h) Astrocytomas, Grade 4 category includes n=3 GBM tumor tissue samples that were determined IDH1 mutant by the ddPCR assay. (f-h) Scatter-dot plots demonstrating the overall distribution (mean±SEM, derived from the average of three technical replicates per study patient) of ddPCR derived: (f) IDH1.R132H mutant copies, (g) IDH1 wild-type copies, and (h) IDH1.R132H MAF % across the three IDH1.R132H mutant glioma subtypes. (f-h) Brown-Forsythe and Welch ANOVA test with multiple comparisons was performed to evaluate the statistical significance of observed differences: (f) Astrocytoma, Grade 2-3 vs Grade 4 (−264.9, p value 0.9371), Astrocytoma, Grade 2-3, vs Oligodendroglioma, Grade 2-3 (−1229, p value 0.0435), Astrocytoma, Grade 4 vs Oligodendroglioma, Grade 2-3 (−963.8, p value 0.3766) (h) Astrocytoma, Grade 2-3 vs Grade 4 (6.794, p value 0.6707), Astrocytoma, Grade 2-3, vs Oligodendroglioma, Grade 2-3 (−17.26, p value 0.0059), Astrocytoma, Grade 4 vs Oligodendroglioma, Grade 2-3 (−24.05, p value 0.0073). (i-j) Kaplan-Meier curves were evaluated among patients with IDH1 mutant glioma by stratifying the patient population using different parameters: (i) glioma subtype, and (j) IDH1.R132H tumor tissue MAF %, cut-off (<38%, >38%). P-value was calculated using Log-Rank test. FIG. 2/panel A, B Created with BioRender.com released under a Creative Commons Attribution-NonCommercial-NoDerivs 4.0 International license (creativecommons.org/licenses/by-nc-nd/4.0/deed.en).

FIGS. 3A-B: Clinical validation of the mt-IDH1_{dx} assay and statistical evaluation of its diagnostic performance using plasma derived Extracellular Vesicle RNA. a) Schematic summarizing the study population (N=133) for blinded validation testing in three independent cohorts. Sensitivity and Specificity calculated for the individual cohorts and the combined study population. Area under curve (AUC) analysis performed to measure assay efficiency. This was done using Wilson/Brown method with results demonstrating an Area 0.8392 and standard error 0.03775 (95% CI: 0.7652-0.9131, p value<0.0001). (b) A comparison of assay the sensitivity, specificity, and accuracy in the study population stratified by histological diagnosis (Astrocytoma, Grade 2-3, Oligodendroglioma, Grade 2-3, and Astrocytoma, Grade 4).

FIGS. 4A-F: Assessment of the prognostic significance of blood-based detection of IDH1.R132H across glioma subtypes using the mt-IDH1aX assay. (a) Kaplan-Meier analysis performed among IDH1 mutant patients using EV RNA IDH1.R132H MAF % cut-off determined by plasma testing. (b) Scatter-dot plot (each data point represents an individual patient; mean of eight technical replicates) demonstrating the overall distribution (mean±SEM, denoted by horizontal line and error bars) of EV RNA IDH1.R132H MAF % across the three IDH1.R132H mutant glioma subtypes: Astrocytoma, Grade 2, 3 (n=46, biological replicates), Oligodendroglioma, Grade 2, 3 (n=19, biological replicates) and Astrocytoma, Grade 4 (n=15, biological replicates). (c-f) Kaplan-Meier curves were evaluated among patients with IDH1 mutant glioma using different approaches: (c) patient stratification by histological diagnosis, (d-f) EV RNA IDH1.R132H MAF % cut-off unique to each subtype (d; Grade 2, Astrocytoma, e; Grade 2-3, Oligodendroglioma, f, Grade 4, Astrocytoma). P-value was calculated using Log-Rank test.

FIGS. 5A-D: The shedding rate of IDH1.R132H mutant tumors varies among the different subtypes of gliomas. (a-b) MA plots comparing the differential enrichment of (a) IDH1.R132H (Mut) expression and (b) IDH1 wild-type (Wt) expression in matched (n=24) tumor tissue derived total RNA versus plasma derived EV RNA. MA plots were created by plotting M value (y-axis; Log₂ ratio=Avg.

Mut/WT, Plasma/Avg. Mut/WT, Tissue) against A value (x-axis; Average, Mut/Wt tissue and Mut/Wt plasma). (a) Patients encircled (red circle demonstrated the highest enrichment in plasma. (c-d) Scatter-dot plots demonstrating the overall IDH1.R132H MAF % distribution in matched (n=24) tumor tissue (c) and plasma samples (d) across multiple variables (Age, Cortical location, Cerebral hemisphere). Kaplan-Meier survival analysis using IDH1.R132H MAF % cut-off as a prognostic indicator in tumor tissue (c: top, right) and plasma (c: bottom, right).

FIGS. 6A-D: Assessing monitoring and surveillance of IDH1.R132H mutant gliomas during the course of clinical therapy. (a-c) Testing of the optimized ddPCR assay in plasma samples (2 ml) collected longitudinally in a cohort of n=8 (labeled, P1-P8) patients. Individual graphs summarizing the EV RNA IDH1.R132H, MAF % (y-axis, mean of 8 replicates) at each clinical time-point (x-axis, time elapsed from baseline detection) with the affiliated radiological findings and clinical outcomes. Study population divided into three groups based on clinical outcomes: (a) disease progression, (b) treatment response, and (c) stable disease. (d) Line plot (third panel, bottom-right) demonstrating the overall trend in measured EV RNA IDH1.R132H MAF % (normalized, described in methods) levels at selected clinical timepoints of treatment: pre-, early- and post-therapy. GTR: Gross total resection, STR: Sub-total resection, Proton RT: Proton therapy, TMZ: Temozolamide, RT: Radiation therapy, PCV: Procarbazine, Lomustine, and Vincristine. FIG. 6/A-C Created with BioRender.com released under a Creative Commons Attribution-NonCommercial-NoDerivs 4.0 International license (creativecommons.org/licenses/by-nc-nd/4.0/deed.en).

FIGS. 7A-C: Assay Design for detection of different nucleic acid analytes and target probe optimization. (a) Schematic depicting the location of IDH1.R132H mutation in exon 4 region (SEQ ID NOs:9 and 10). Probes and primer combinations varying in nucleic acid detection (RNA, DNA) and amplicon size (49 bp-62 bp) illustrated on the nucleotide sequence (Fwd49: AGTGGATGGGTAAAACC (SEQ ID NO:1); Rev49: TCATGCTTATGGGGATC (SEQ ID NO: 11); Rev 62: complementary to GATCAATACAGAGCAAC (SEQ ID NO:3); Mutant Probe 1: TATCATCATAGGTCA: (SEQ ID NO:12); Probe 2: TATCATCATAGGTCAT (SEQ ID NO:13); Probe 3: TATCATCATAGGTCATC (SEQ ID NO:14); Probe 4: TCATAGGTCATCATGCTTA (SEQ ID NO:15). (b) Four different Mutant probe designs (Probes 1-4, SEQ ID NOs:12-15) highlighted, with corresponding 1-D amplitude (mutant channel; top panel, wild-type channel; middle panel)) and 2-D amplitude (bottom panel) droplet digital PCR (ddPCR) plots depicting the impact on overall signal separation and mutant, wild-type probe cross-reactivity. (c) Comparison and optimization of the selected probe (Probe 4, SEQ ID NO:15, panel c) chemistry (TaqMan Minor Groove Binder (MGB) versus locked nucleic acid (LNA)) across a range of annealing temperatures (49.0-54.0) ° C.

FIGS. 8A-I: Reverse transcription (RT) and Droplet Digital PCR (ddPCR) optimization summary. (a) ddPCR 1-D amplitude plot (temperature gradient) depicting an overall better separation of IDH1.R132H signal at lower annealing temperature (<55° C.). (b) Further comparison of the IDH1.R132H copy number using selected assays (left panel) with highest copy number detected at 51° C. (right panel, n=2, technical replicates). (c) Workflow (left panel) to compare the signal recovery of equal spike-in amounts (0.63 ng-2.50 ng) of IDH1.R132H RNA/DNA in an EXOLUTIONPLUS™ (kit for isolating EV RNA and DNA) background using healthy plasma. Bar-graph (right panel) quantifying the average mutant copy number (n=2 replicates). (d) Experimental design (left panel) to investigate the inhibitory effect of DNA at reverse transcription (RT) step, using tumor tissue (n=1) derived varying spike-in DNA concentrations (2.5 ng-10.0 ng) in a constant RNA background (10.0 ng). Box-plots (floating bars; min to max, line at mean) (right panel, n=3 replicates) demonstrating the reduction in IDH1.R132H copy number with DNA addition at the RT step. (e) Stacked bar-plot (left panel) demonstrating the IDH1.R132H copy number detected using three RT (left panel) enzymes (SS III VILO; SuperScript III VILO, SS IV VILO; Superscript IV VILO, SS IV; SuperScript IV) at three different RNA inputs (n=3 technical replicates). Scatter-plot (right panel) comparing the IDH1.R132H copy number detected using selected assays, following preamplification with two supermix (polymerase) protocols (n=3, technical replicates). ANOVA with multiple comparisons and unpaired t tests were performed to compare the means across two assays and protocols respectively (right panel). (f) Bar-graph (left panel, n=2 technical replicates) demonstrating complete elimination of IDH1 wt signal using Wt3 (vs. Wt1) during preamplification of tumor tissue derived RNA. Violin plot (right panel) comparing the IDH1.R132H EV RNA MAF % in n=2 patient plasma samples using two different concentrations (200 nm, 400 nm) of Wt3, (n=5 technical replicates). ANOVA was run to compare the mean copy number difference in P1 and P2 with the addition of Wt3. (g-i) Assessment of reproducibility of IDH1.R132H EV RNA detection in plasma (2 ml) in a cohort of n=6, IDH1.R132H mutant patients. For each patient two runs (R1; replicate 1, R2; replicate 2, n=8 technical ddPCR replicates) were performed at the same clinical time-point. Violin plots illustrate the variation in: IDH1.R132H copies (g), IDH1 wild-type copies (h), and IDH1.R132H MAF % (i). Panels C-D Created with BioRender.com released under a Creative Commons Attribution-NonCommercial-NoDerivs 4.0 International license (creativecommons.org/licenses/by-nc-nd/4.0/deed.en).

FIGS. 9A-D: Clinical Validation of mt-IDH1ₓ Droplet Digital PCR (ddPCR) assay in patient plasma samples. (a-d) Optimized mt-IDH1_{dx} assay was evaluated for detection of IDH1.R132H EV RNA in plasma isolated from a study population (N=133) divided into three cohorts. (a) Scatter dot plot demonstrating the IDH1.R132H MAF % distribution in histopathology confirmed IDH1.R132H mutant (blue, n=80), IDH1 wild-type (green, n=44) samples, and age matched healthy controls (red, n=9). Horizontal bars indicate mean and error bars represent standard error (SEM). For each patient and control sample, n=8 replicates were performed at the ddPCR step. Brown-Forsythe and Welch ANOVA tests were performed to compare the means across the study population groups. (b-c) Scatter dot plots illustrating the overall distribution of: (b) IDH1.R132H copy number, (c) IDH1 wild-type copy number, in IDH1.R132H mutant plasma samples (n=80) stratified by glioma subtype (Astrocytoma, grade 2-3; n=50, Oligodendroglioma, grade 2-3; n=19, Astrocytoma, grade 4; n=11). (b-c) Horizontal bars indicate mean and error bars represent standard error (SEM). (d) Statistical calculation using Youden index to determine the cut-off for the “number of positive ddPCR replicates” to classify a sample as “positive” versus “negative” for presence of IDH1.R132H mutation.

FIG. 10. Exemplary workflow for an assay as described herein.

### DETAILED DESCRIPTION

Liquid biopsy, defined as sampling and analysis of biofluids (plasma, cerebrospinal fluid, urine, saliva) has emerged as a promising platform for rapid, minimally invasive diagnosis of a range of cancers including gliomas^{10,11,12}. This is achieved by isolation and downstream analysis of a range of circulating tumor tissue derived analytes closely reflecting the epitranscriptome profile of tumor cell of origin: extracellular vesicles (EVs), circulating tumor cells (CTCs), cell-free DNA (cfDNA), and circulating RNA. The present inventors previously optimized and developed highly sensitive (>75%) and specific (>90%) blood and CSF based assays for detection of pivotal DNA (TERT C228T/C250T¹³) and RNA (EGFRvIII^{14,15}) based alterations prevalent in different glioma subtypes.

Expanding our efforts to IDH1.R132H detection, the present inventors previously utilized the technology of BEAMing (beads, emulsion, amplification, magnetics) PCR for reliable detection of IDH1.R132H mutant EV RNA transcripts in CSF of patients with gliomas¹⁶. Described herein is an assay, one embodiment of which is a Droplet Digital PCR (ddPCR) assay called mt-IDH1_{dx}, for robust detection of IDH1.R132H in patient plasma derived EV RNA. To evaluate the assay performance in our study population (N=133), blinded validation testing was performed across three independent cohorts. Baseline Plasma testing from IDH1.R132H patients (n=80), IDH1 wild-type gliomas (n=44), and age matched healthy controls (n=9) demonstrated clinically relevant diagnostic sensitivity and specificity. Finally, the utility of the proposed blood-based in longitudinal disease monitoring and surveillance was shown in (n=8) patients with IDH1.R132H alteration.

Historically, glioma classification relied largely on histology and immunohistochemistry¹⁷. Integration of molecular alterations, specifically IDH1.R132H, was first introduced in 2016¹⁸ WHO Classification of CNS tumors and later expanded in 2021¹⁹ after its breakthrough discovery in gliomas in 2008². Accurate diagnosis of this unique molecular variant is critical in determining the clinical and surgical management of an expansive lesion on magnetic resonance image (MRI). Typically, hyperintensity on T2-weighted images and T2-fluid-attenuated inversion recovery sequences (FLAIR) features are highly suggestive of a likely IDH1.R132H mutant glioma^{20,21}. However, definitive diagnosis still requires the histopathology assessment of tumor tissue.

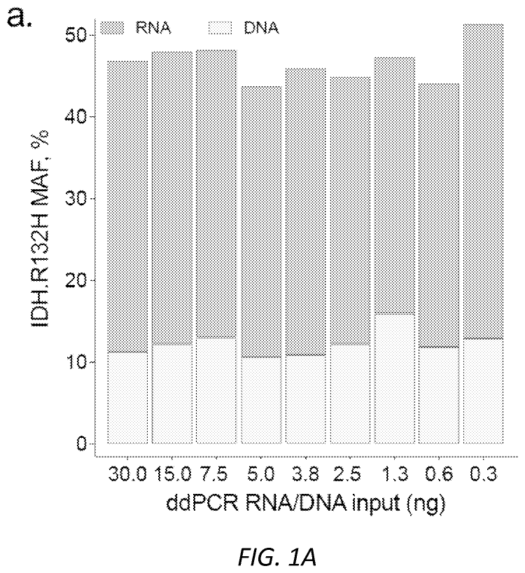

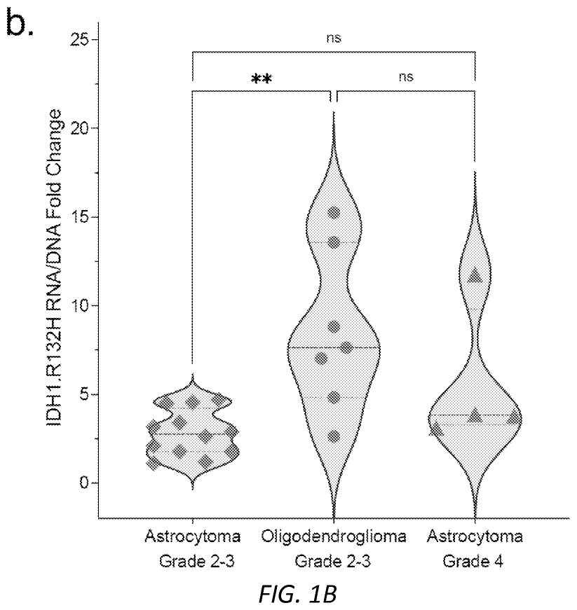

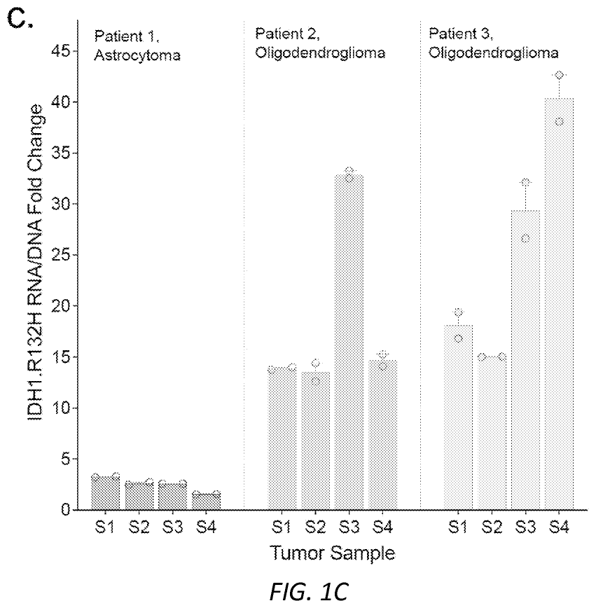

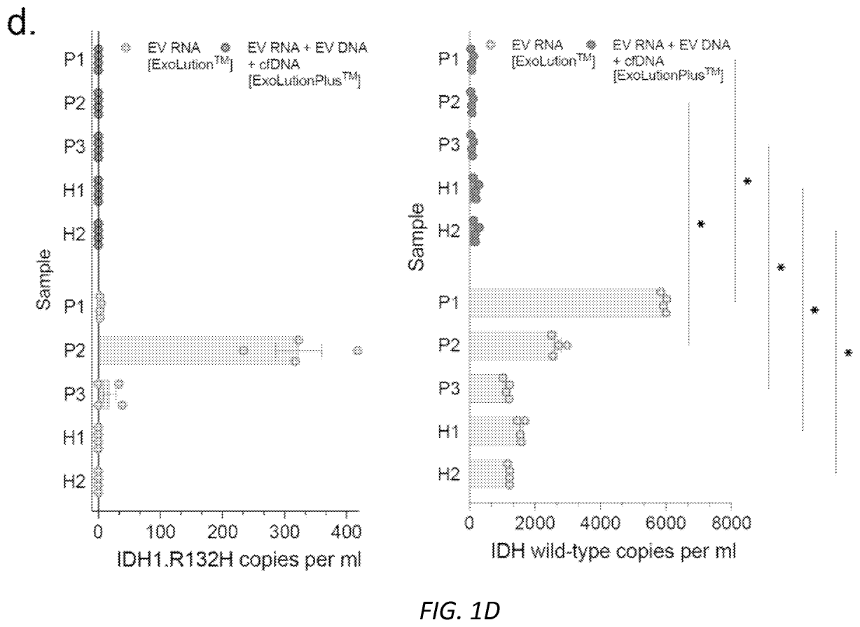

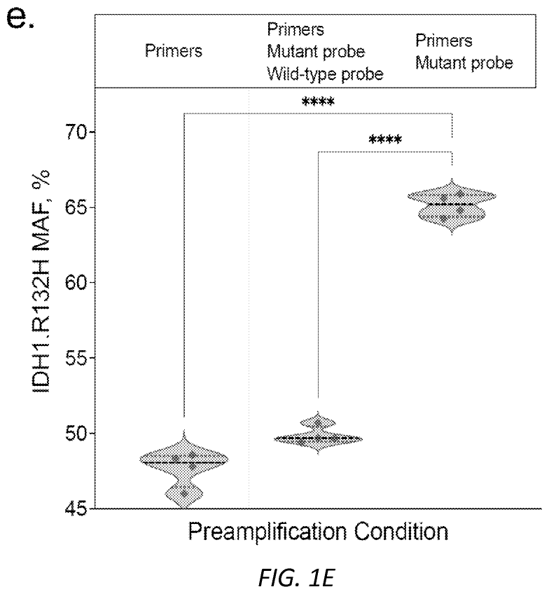

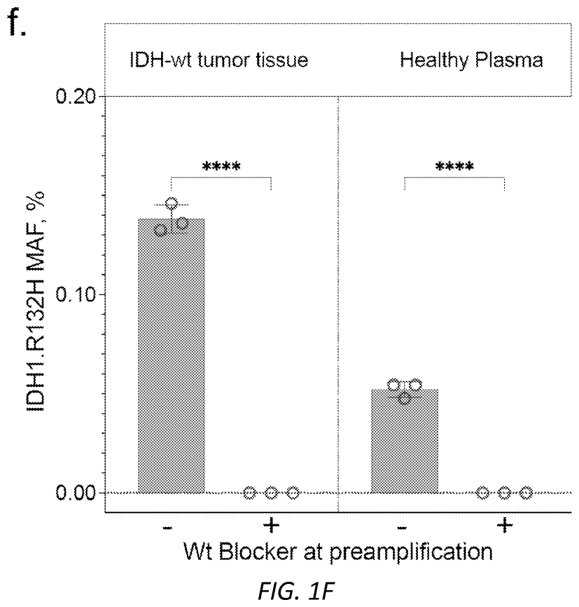

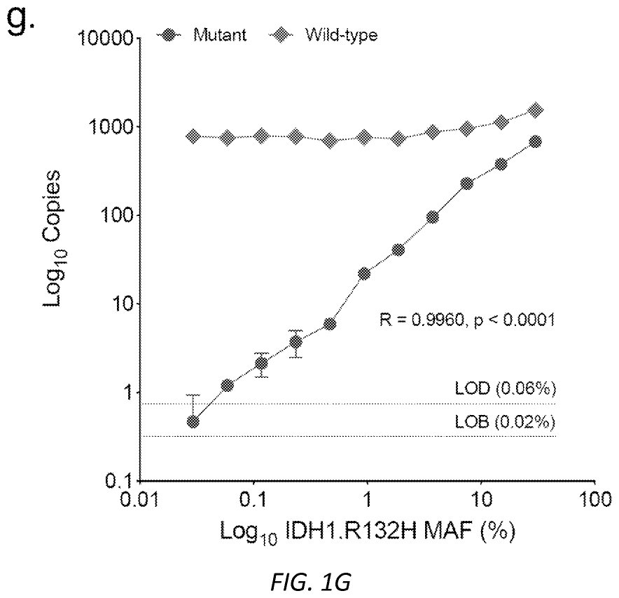

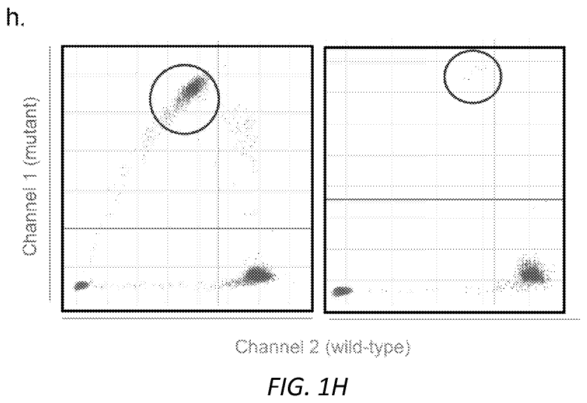

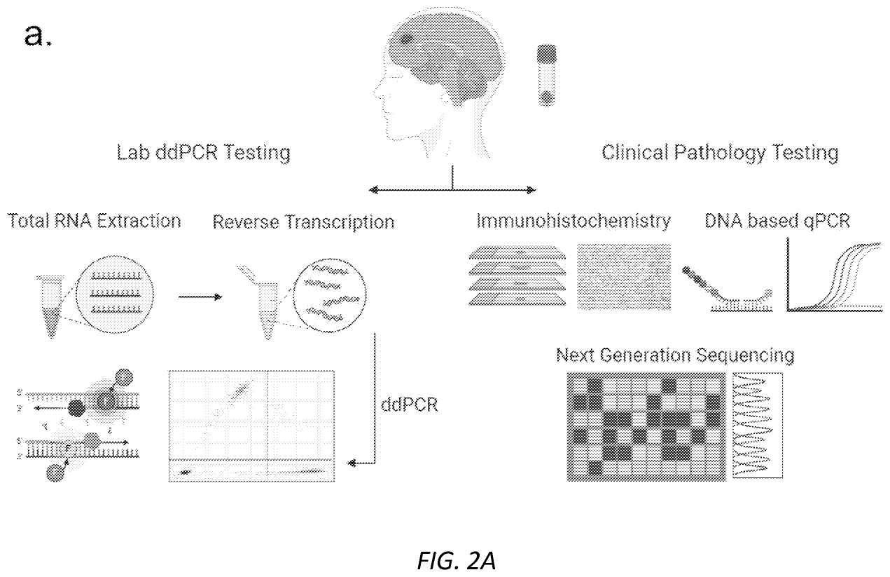

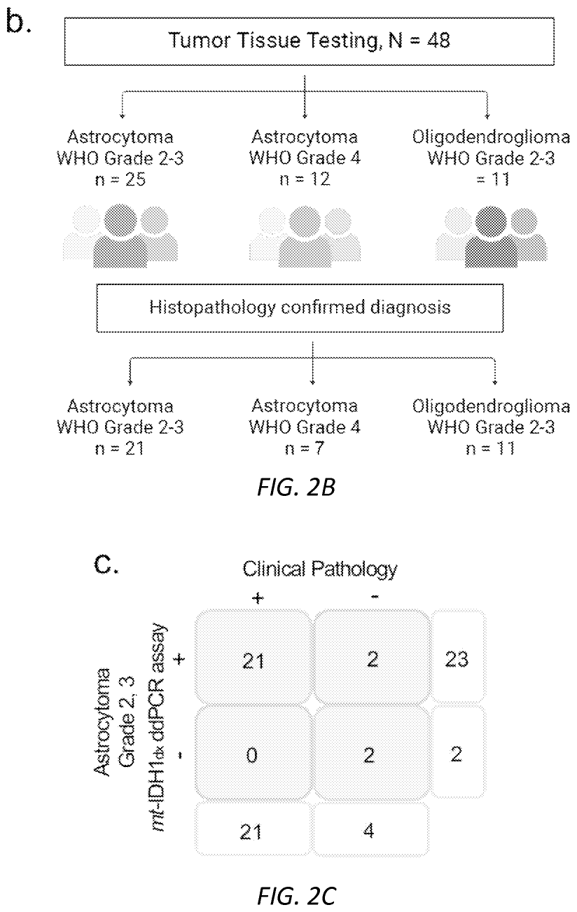

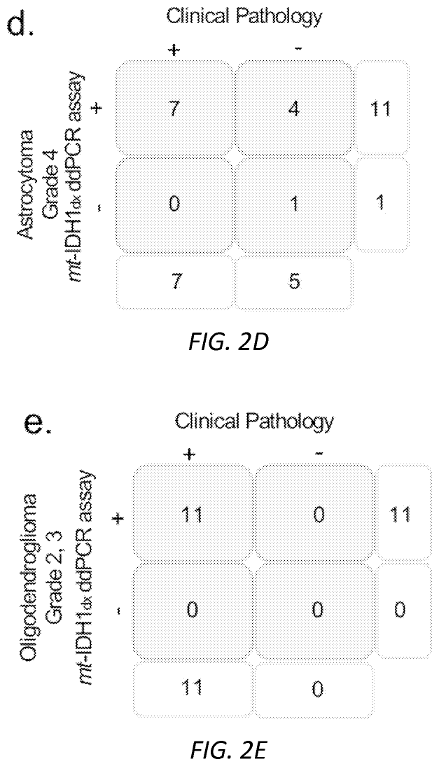

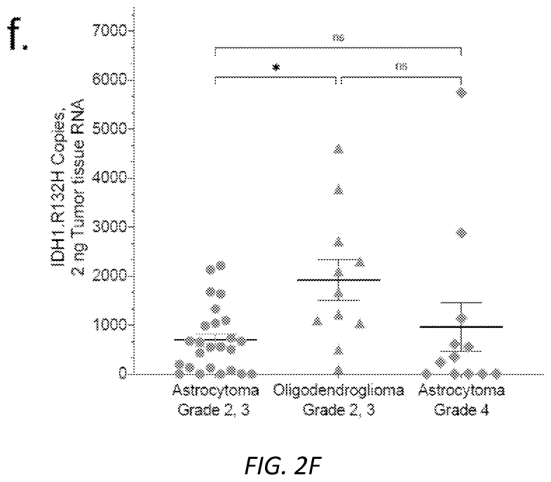

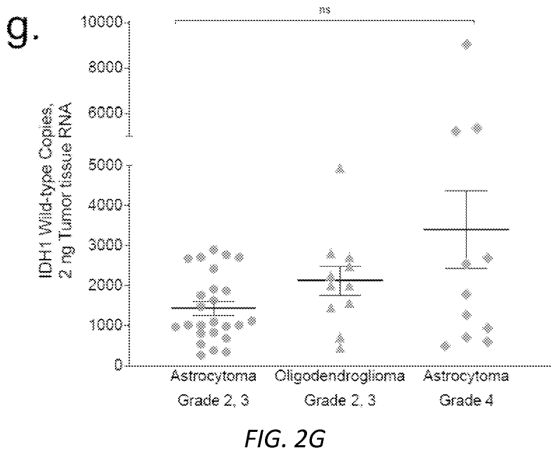

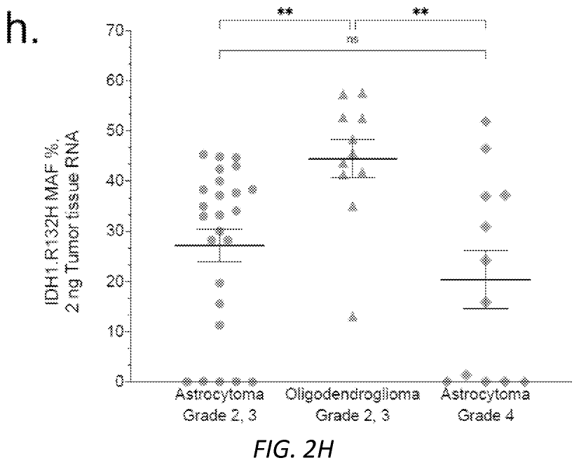

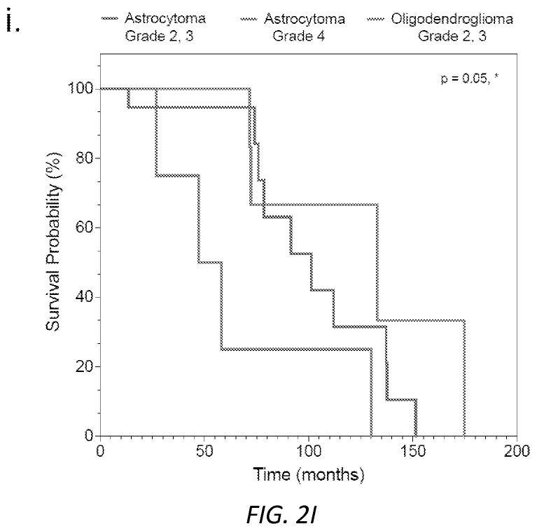

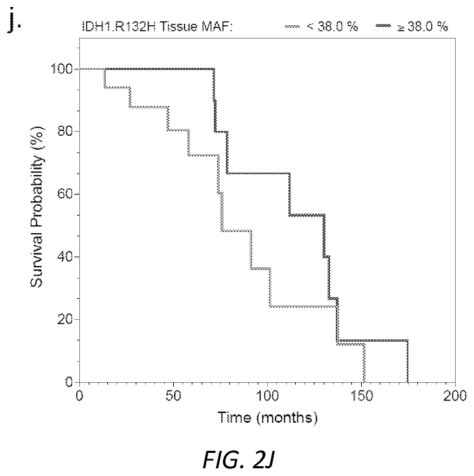

Previous work in liquid biopsy (plasma, CSF) based assessment of IDH1.R132H mutational status has predominantly utilized cell free DNA (cfDNA)^{22, 26} as the candidate biomarker with an overall limited sensitivity (25%-63%)²²⁻²⁶. Results from testing shown herein demonstrated that an RNA based assay is more sensitive than DNA across focal and spatial tumor tissue analysis of different IDH1 mutant glioma subtypes. Interestingly, oligodendrogliomas (n=7) and grade 4 astrocytomas (n=4) showed a wider distribution of the IDH1 mutant and wild-type RNA:DNA ratios, with astrocytomas, grade 2-3 (n=12) showing a more homogeneous distribution (FIG. 1b-c, FIG. 2f-g). This is likely a reflection of differences in tumor biology, including the extent of tumor infiltration²⁷, as well as a wider age range distribution in oligonderogliomas than astrocytomas as previously reported²⁸. Importantly, in the present cohort, astrocytomas also had a lower age range (44.0) and coefficient of variation (22.6%) compared to oligodendrogliomas (age range: 53, coefficient of variation: 30.7%).

mt-IDH1_{dx} assay performance was evaluated in comparison to the traditional gold standard clinical pathology testing. Overall, in the present study there were n=2 tissue and n=3 plasma samples that were in discordance with histopathology assessment. Notably, using our mt-IDH1_{dx} assay we identified the IDH1.R132H mutation in a subset of astrocytoma patients originally classified as IDH1.R132H wild-type (n=2; expressing IDH1.R132C or IDH1.R132S) based on pathology testing. This discordance can be attributed to several factors: i) difference in sensitivity between ddPCR and IHC methods, ii) preferential use of cost-effective (but less sensitive) testing techniques (IHC, DNA qPCR) in patients≥55 years of age with radiological suspicion of grade 4 astrocytoma (previously classified as Glioblastoma) due to low pre-testing probability of the presence of IDH1 mutation²⁹, iii) tissue sampling errors (insufficient, non-viable), and iv) intra-tumoral spatial heterogeneity. Presence of multiple, genetically distinct subclones with diverse evolutionary potential has been well described for IDH1 wild-type GBM^{30,31} and more recently, IDH1.R132H mutant glioma³²⁻³⁴. Astrocytomas (grade 3-4) harboring such “subclonal IDH1 mutation” (defined as tumor cell fraction with IDH mutation≤0.67) have also been reported³⁵. Hence, sensitive plasma testing can facilitate a more comprehensive determination of mutational status as shown by the confirmation of the IDH1mt signal in tumor tissue (following positive signal in plasma). Considering the challenges encountered in standard clinical testing, the tissue and blood-based assay described herein provides a feasible platform for rapid and sensitive diagnostics, overcoming the limitations posed by disease heterogeneity and technical limitations.

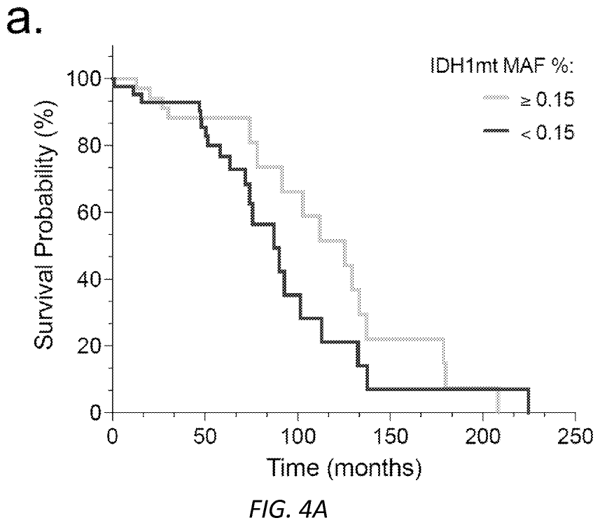

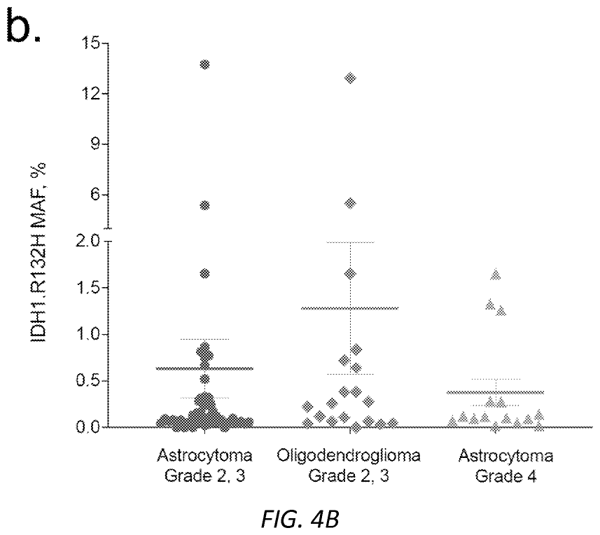

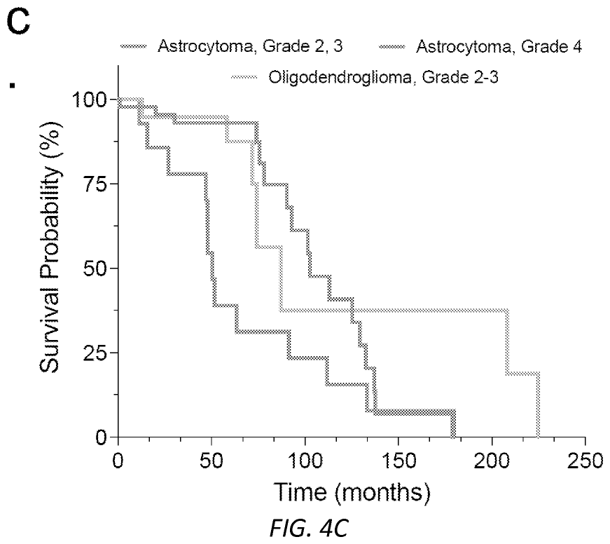

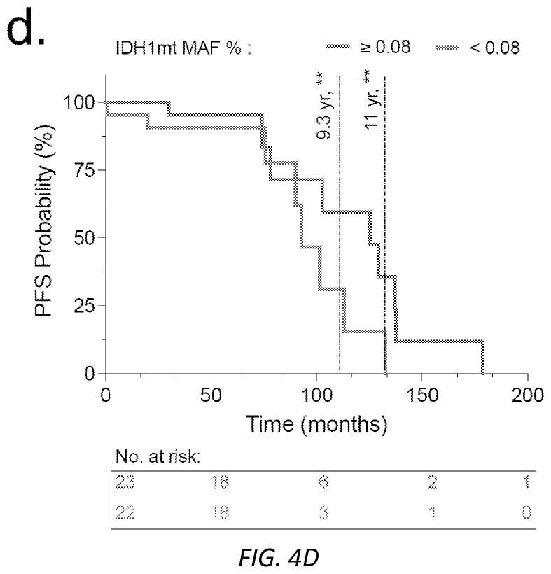

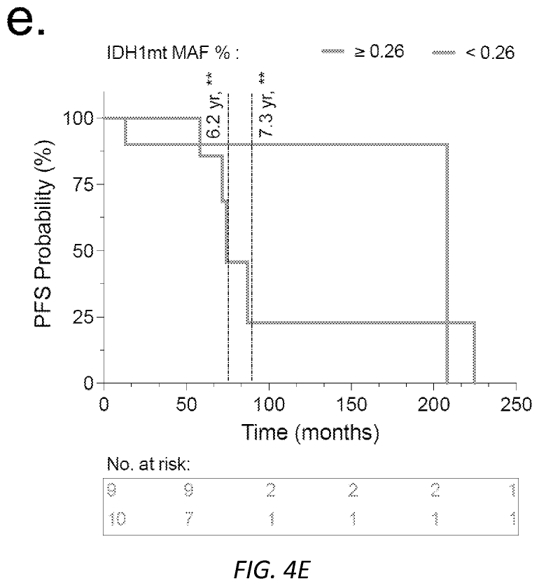

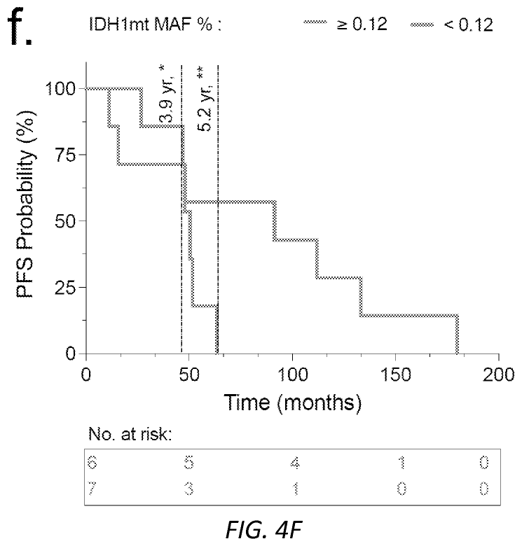

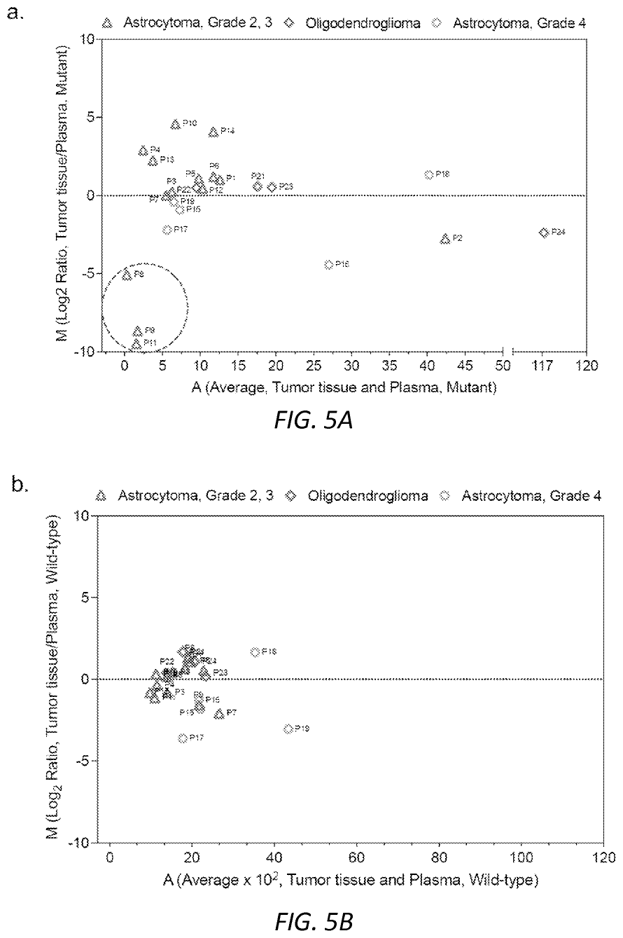

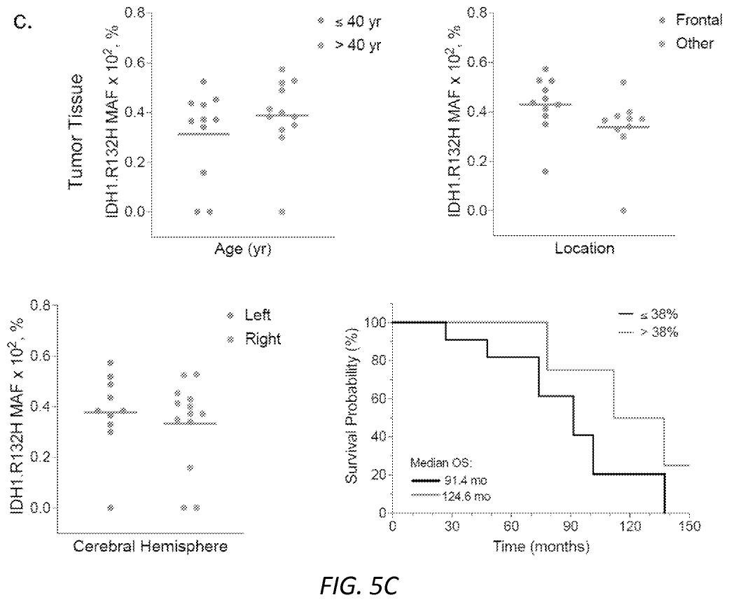

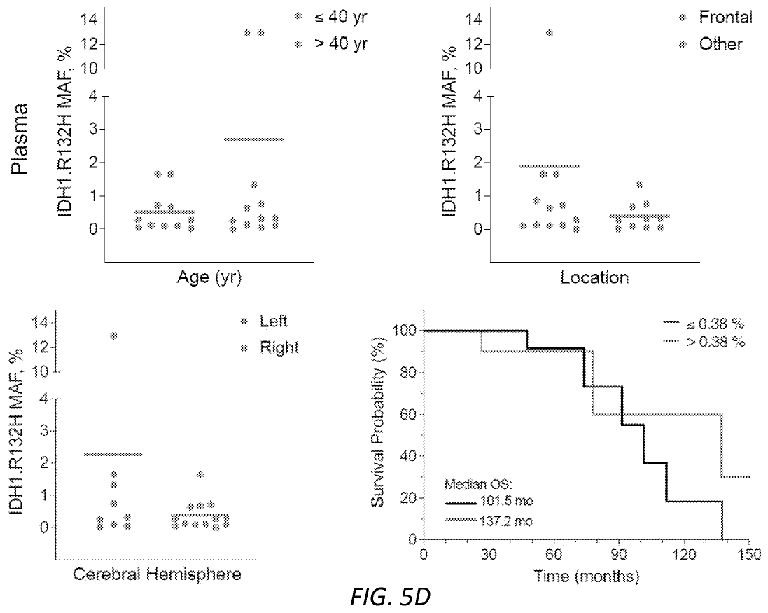

Here the correlation between IDH1.R132H MAF (tumor tissue, plasma) and multiple disease parameters was also analyzed. Notably, there were consistently similar trends in independent tumor tissue (FIG. 2) and plasma (FIG. 4) cohorts. A striking observation was that high tissue and plasma IDH1.R132H MAF served as a positive prognostic factor, associated with longer OS across all subtypes. While it is well established that oligodendroglioma is associated with improved overall survival³⁶, as reported herein the improved OS correlated with increased plasma MAF and tumor tissue MAF. Furthermore, IDH1.R132H MAF distribution in matched and unmatched plasma/tissue samples highlighted key trends, with higher MAF seen in the following variables: frontal location, left hemisphere, and age≥40 yr. Finally, tissue/plasma copy enrichment analysis indicated variation in glioma solid tumor shedding rate across different subtypes. While the measured IDH1wt expression was consistent in both tissue (total RNA) and plasma (EV RNA), IDH1mt expression showed considerable variation, highlighting further the significance of investigating heterogeneity of IDH1.R132H mutant tumor biology, specifically in astrocytomas. Additionally, tumors with higher shedding levels (low grade astrocytomas) were associated with overall improved survival as compared to lower shedding tumors (high grade astrocytomas; FIG. 5). A similar trend has been observed for other cancers like hepatocellular carcinoma³⁷, melanoma³⁸, pancreatic and colon cancer³⁹, and in vivo glioma models⁴⁰.

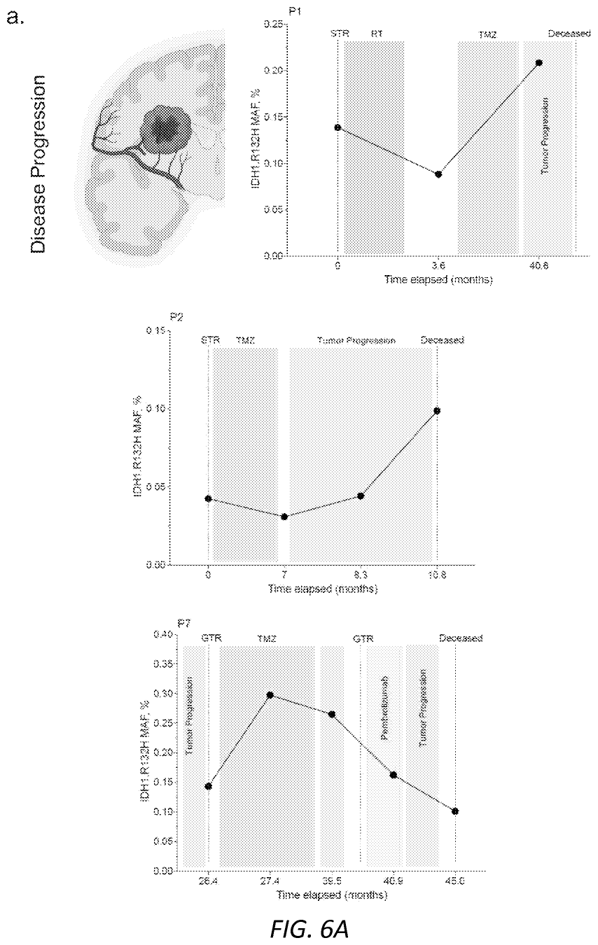

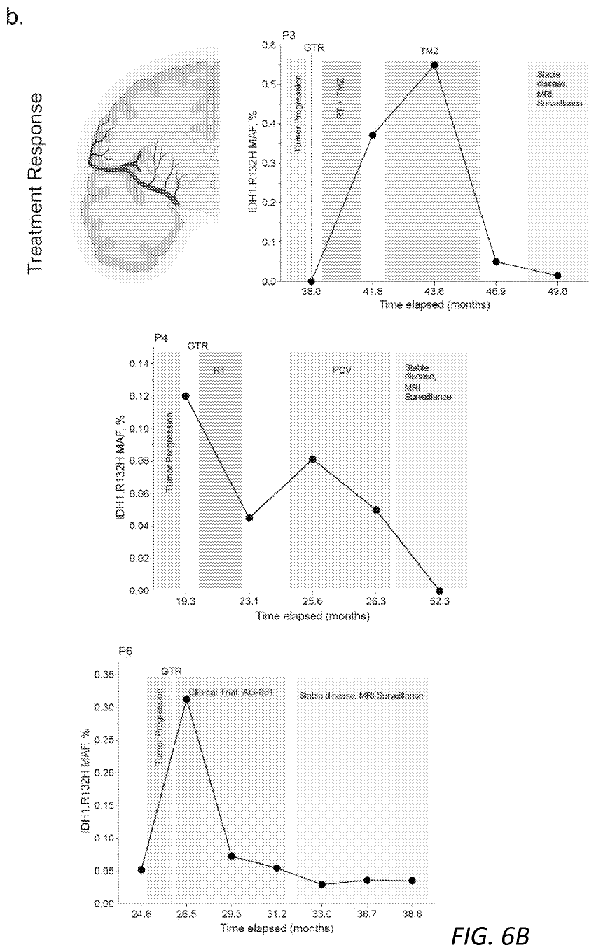

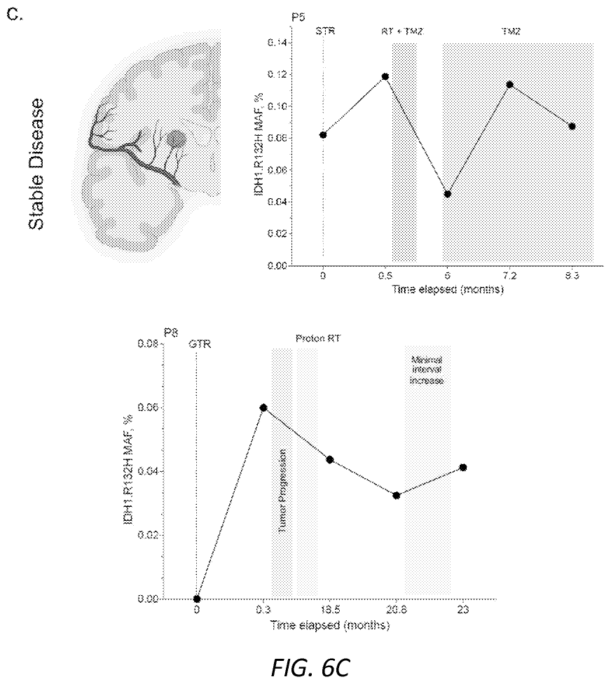

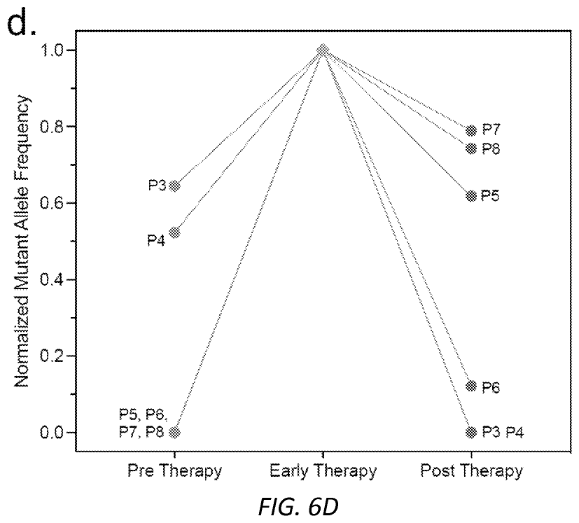

Lastly, the blood-based assay was analyzed in longitudinal settings to determine its utility in disease monitoring, predicting treatment response, and surveillance. Overall, results demonstrated concordance with the radiological imaging findings and/or tissue biopsy assessment across different clinical outcomes: tumor progression, treatment response, stable disease. Interestingly, in two patients (P3, P8) the assay was not able to detect the mutation at baseline testing. P3 had an initial diagnosis of Astrocytoma, Grade 2 with tumor volume below 1 cm³. Based on our correlation analysis (FIG. 5), IDH1.R132H MAF tends to be lower in non-frontal, right sided lesions, which is where this patient's was located. Furthermore, the lesion measured 13×23 mm in maximum axial dimension. Interestingly, as per clinical pathology tissue assessment, the lesion was IDH1 wild-type by IHC but later confirmed IDH1mt by Snapshot analysis. However, following tumor progression the assay detected IDH1mt in plasma with levels showing correlation to the disease course. A second patient (P8) demonstrated a similar trend where the IDH1mt signal was not detected at the first time point (pre-surgery) but was detected at high levels at a later time-point. Similarly, P8 had a low disease burden at initial diagnosis but progressed following initial surgical resection as shown by an enhancing radiological signal and worsening cognitive function. Another key experimental observation was the overall trend in measured EV RNA IDH1.R132H MAF (normalized) across different patients receiving TMZ and PCV (FIG. 6d). The initial rise followed by decline in levels provides insight into the tumor shedding mechanism in response to treatment and hence, interpretation of increased tumor derived analytes in the bloodstream. It has been shown that EVs secreted by melanoma cells in response to chemotherapy can modulate and predict the tumor response to alkylating drugs. However, this has not been investigated in the realm of gliomas. It was hypothesized that EV RNA cargo can be serially quantified to not only determine but also predict treatment response. For instance, in the present analysis patients with complete treatment response (radiological evidence; P3, P4, P6) had a much larger decline in MAF following initial rise as compared to patients with radiological evidence of residual disease (P5, P7, P8). Interestingly, while the MAF did correlate with overall outcomes in treatment naive glioma patients, the pattern was different in recurrent tumors undergoing treatment^{41,42}. These differences are likely a reflection of the evolution in tumor biology, mutational landscape, tumor microenvironment, and blood-brain-barrier disruptions^{41,42}. Important to consider is also the time period (often up to years) between initial diagnosis and recurrence. Importantly, this current data on IDH1.R132H as well as previous work on TERTp¹³ and EGFRvIII¹⁴, consistently show a higher MAF and/or copy number at final tumor recurrence time point as compared to baseline. The glioma treatment may alter the shedding rate in the plasma of glioma patients, shifting its association from overall survival to the treatment course of the disease. Recent work also highlights a spike in the shed analyte into biofluids (CSF and plasma) at the early therapy timepoint and underscores the value of monitoring treatment course using liquid biopsy especially in targeted therapeutics, including CAR T Cell treatment⁴³.

In summary, the field of liquid biopsy diagnostics and theranostics is evolving rapidly. Gliomas, a diverse group of tumors in the central nervous system, exhibit a wide range of molecular and epigenetic characteristics. The IDH1.R132H mutation, present in both low-grade and high-grade gliomas, stands out as a distinct alteration with significant prognostic implications. Described herein is a highly sensitive platform designed for rapid testing, offering multiple clinical applications, including i) diagnosis, ii) prognostication, iii) monitoring of treatment response, and iv) longitudinal disease monitoring and surveillance.

### Assays

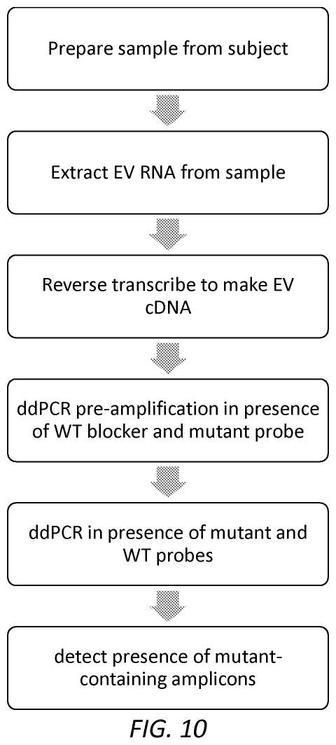

The assays provided herein generally include the steps described in FIG. 10. The methods described herein involve use of a pair of amplification primers and detection probes to detect IDH1.R132H mutations with high sensitivity and high specificity in a sample from a subject. Subjects who can be evaluated or treated in the present methods include humans. The sample can comprise a biofluid, e.g., plasma or blood, saliva, urine, and/or cerebrospinal fluid (CSF). Alternatively, the sample can comprise tissue or cells, e.g., tumor tissue or cells, e.g., obtained from a biopsy or surgical resection.

### Sample Preparation and Processing

A sample can be processed or prepared using nucleic acid extraction conditions provided herein to achieve higher concentrations of IDH1.R132H sequences from the same amount of starting material than can be achieved using other conditions. Such nucleic acid extraction conditions involve isolation of EV-associated ribonucleic acids (EV RNA) from the biological sample using any suitable method known in the art or described herein. A number of methods and commercially available kits can be used to isolate EV RNA, including bead-based isolation, precipitation methods or ultracentrifugation.

In some embodiments, nucleic acid extraction conditions can comprise contacting the biological sample with a capture surface under conditions sufficient to retain the microvesicle fraction on or in the capture surface, wherein the capture surface comprises three membranes that are positively charged and finctionalized with quaternary ammonium; lysing the microvesicle fraction while the microvesicles are on or in the capture surface; and extracting the nucleic acids from the microvesicle fraction. See, e.g., WO2014107571A1. The methods can also include isolating microvesicles comprising exosomes from the biological sample by one or more centrifugation procedures to pellet the microvesicles from the biological sample and generate isolated, pelleted microvesicles comprising exosomes, optionally wherein none of the centrifugation procedures are performed at a speed exceeding 20,000 g; and extracting nucleic acids from the isolated, pelleted microvesicles comprising exosomes, wherein the extracted nucleic acids comprise RNA. See, e.g., WO2012064993A1. The conditions can comprise contacting the biological sample with an anion exchange membrane or anion exchange bead and polyethylene glycol, contacting the membrane or the bead with a guanidine thiocyanate-based elution buffer, which releases the nucleic acids from the membrane or beads to produce a homogenate, and contacting the homogenate with a silica-based solid surface, thereby extracting the nucleic acids from the homogenate. In some embodiments, nucleic acid extraction conditions further comprise adding a protein precipitation buffer to the homogenate prior to extraction of the nucleic acid from the homogenate.

In some embodiments, the beginning sample comprises the entire plasma RNA extract from a 2 ml whole blood sample.

A further non-limiting example of nucleic acid extraction conditions that can be used as provide herein are provided in U.S. Pat. No. 10,808,240, the relevant disclosures of which are herein incorporated by reference for the purposes and subject matter referenced herein. Alternatively, or in addition to, nucleic acid extraction conditions provided herein can be achieved using a commercially available kit such as EXOLUTION (Exosome Diagnostics), ThermoFisher total EV, Systems Bio EVery EV, Izon qEV columns., See, e.g., Hsia et al., Adv Biol (Weinh). 2024 January; 8(1):e2300233.

The EV RNA is then subjected to reverse transcription to make EV cDNA using methods known in the art, e.g., as described herein.

In cases where tumor tissue or cells are used, the methods do not need to include isolating EVs, but can be performed on cDNA made by reverse transcription from RNA obtained directly from the tissue or cells.

### Pre-Amplification

Methods described herein can include a step of pre-amplification of a mutant region of an IDH in cDNA made from EV RNA from a subject sample using a pair of amplification primers, i.e., a forward primer and a reverse primer. An example of a pair of amplification primers comprising a forward primer and a reverse primer flanking (i.e., one on either side) a mutated portion of an IDH gene sequence, e.g., Forward mRNA primer (5′-AGTGGATGGGTAAAACC-3′ (SEQ ID NO:1)), and Reverse mRNA primer (complementary to 5′-GATCAATACAGAGCAAC-3′ (SEQ ID NO:3, e.g., 5′-GTTGCTCTGTATTGATC-3′ (SEQ ID NO:2)). Alternatively, the reverse primer can comprise SEQ ID NO:18 or 19.

In some examples, the forward primer comprises a nucleotide sequence that is at least 80% identical, at least 85%, at least 90%, at least 91%, at least 92%, at least 93%, at least 94%, at least 95%, at least 96%, at least 97%, at least 98%, or at least 99% identical to SEQ ID NO: 1. In some embodiments, the forward primer comprises SEQ ID NO: 1. In some embodiments, the forward primer is SEQ ID NO: 1.

In some examples, the reverse primer comprises a nucleotide sequence that is at least 80% identical, at least 85%, at least 90%, at least 91%, at least 92%, at least 93%, at least 94%, at least 95%, at least 96%, at least 97%, at least 98%, or at least 99% identical to SEQ ID NO: 2, 18, or 19. In some embodiments, the reverse primer comprises SEQ ID NO: 2, 18, or 19. In some embodiments, the reverse primer is SEQ ID NO: 2.

It should be understood that the forward primer and/or the reverse primer can comprise a modified nucleotide such as those known in the art or described herein. Modified nucleotides include, but are not limited to, nucleotides comprising a backbone modification, a base modification, and/or a sugar modification. Non-limiting examples of backbone modifications include phosphorothioate modifications, methylphosphonate modification, phosphoramidate modifications, and locked nucleic acid (LNA) backbone modifications. Non-limiting examples of base modifications include substituted purines and pyrimidines. Non-limiting examples of sugar modifications include 2′-O-alkylated or 2-fluorinated ribose and arabinose. Other such modifications are well known to those of skill in the art.

In some embodiments, the mutant primer comprises at least 1, at least 2, at least 3, at least 4, at least 5, at least 6, at least 7, at least 8, at least 9, at least 10, or more modified nucleotides. In some embodiments, the wild-type primer comprises at least 1, at least 2, at least 3, at least 4, at least 5, at least 6, at least 7, at least 8, at least 9, at least 10, or more modified nucleotides.

The pre-amplification is preferably performed in the presence of a wild-type blocking oligonucleotide, that binds preferentially to the wild type but not the mutant sequence. Exemplary blocking oligos are described herein, including Wt1 (5′-CATAGGTCTCATGCTTA-3′ (SEQ ID NO:6)), Wt2 (5′-CCCCATAAGCATGAGACCTATG-3′ (SEQ ID NO:7)), Wt3 (5′-CCCCATAAGCATGACGACCTATG 3′ (SEQ ID NO:8)). Preferably, the Wt3 sequence with a 3′ phosphorylation modification (CCCCATAAGCATGACGACCTATG/3Phos/-3′, SEQ ID NO:8) is used. Preferably, the blocker is modified at the 3′ end to prevent extension, e.g., with 3′ phosphorylation. In some embodiments, the wild-type blocking oligonucleotide comprises a nucleotide sequence that is at least 80% identical, at least 85%, at least 90%, at least 91%, at least 92%, at least 93%, at least 94%, at least 95%, at least 96%, at least 97%, at least 98%, or at least 99% identical to SEQ ID NO: 8. In some embodiments, the forward primer comprises SEQ ID NO: 8. In some embodiments, the forward primer is SEQ ID NO: 8.

Exemplary preamplification thermocycling conditions include the following: initial enzyme activation at 95° C. (51% ramp) for 2-15 minutes, e.g., 10 minutes, then 8-20 cycles, e.g., 10 cycles, of denaturation at 94° C. (51% ramp) for 30 seconds, and annealing/extension at 51.0° C. for 1 minute, followed by enzyme inactivation at 98° C. for 10 minutes.

ddPCR Amplification and Detection

The pre-amplified EV cDNA, or the tumor or cell-derived cDNA, is then subjected to ddPCR amplification using known methods (see, e.g., Mazaika and Momsy, Curr Protoc Hum Genet. 2014 Jul. 14; 82:7.24.1-7.24.13).

Preferably, the ddPCR is performed in the presence of an IDH mutant probe and an IDH Wild-type probe. The probes are preferably labeled on the 5′ end withou a fluorophore and on the 3′ end with a corresponding quencher that absorbs the emission from the fluorophore. During the ddPCR extension step, when the DNA polymerase reaches the probe, the polymerase's 5′ to 3′ exonuclease activity cleaves the probe to release the fluorophore from the quencher; fluorescence emitted can then be detected upon excitation with the corresponding wavelength. Exemplary modified sequences include the following: mutant probe, 5′-/fluor/TCATAG+GTC+ATC+ATG+CTTA/quencher/-3′(SEQ ID NO:4, and WT probe, 5′-/fluor/TCATAG+GTC+GTC+ATG+CTTA/quencher/-3′ (SEQ ID NO:5). For example, the 5′ end can be labeled with a fluorophore such as FAM (single isomer derivative of fluorescein), HEX (hexachloro-fluorescein), YAK (YAKIMA YELLOW), Cy3, Cy5, or Texas Red, and the 3′ end with a quencher such as IOWA BLACK FQ, IOWA BLACK RQ, ZEN Quencher, and TAMRA. The mutant and WT probes should preferably be labeled with different fluorophores to enable detection of both.

In some embodiments, the mutant detection probe comprises a nucleotide sequence that is at least 80% identical, at least 85%, at least 90%, at least 91%, at least 92%, at least 93%, at least 94%, at least 95%, at least 96%, at least 97%, at least 98%, or at least 99% identical to SEQ ID NO: 4 or 24. In some embodiments, the mutant detection probe comprises SEQ ID NO: 4 or 24. In some embodiments, the mutant detection probe is SEQ ID NO: 4.

In some embodiments, the wild-type detection probe comprises a nucleotide sequence that is at least 80% identical, at least 85%, at least 90%, at least 91%, at least 92%, at least 93%, at least 94%, at least 95%, at least 96%, at least 97%, at least 98%, or at least 99% identical to SEQ ID NO: 5 or 25. In some embodiments, the wild-type detection probe comprises SEQ ID NO: 5 or 25. In some embodiments, the wild-type detection probe is SEQ ID NO: 5.

### Applications of Methods for Detection of IDH1 Mutations

The assay methods described herein can be used for both clinical and non-clinical purposes. Some examples are provided herein. For example, the methods described herein can be applied for evaluation of cancer, e.g., diagnosis or prognosis of a cancer. Evaluation can include identifying a subject as being at risk for or having a cancer as described herein. Evaluation can also include monitoring treatment of a cancer, such as evaluating the effectiveness of a treatment for a cancer, and/or monitoring reoccurrence of a cancer. Methods described herein can also be applied for non-clinical applications such as for research purposes.

The cancers that can be diagnosed or evaluated using the present methods include cancers in which the IDH1 R132H mutation have been detected, e.g., gliomas and myeloid malignancies such as acute myeloid leukemia (AML) and myelodysplastic syndromes (MDS), chondrosarcoma, cholangiocarcinoma, astrocytoma, secondary glioblastoma (diffuse astrocytoma), ependymoma, primary glioblastoma, cartilaginous and bone tumors, intrahepatic cholangiocarcinoma, acute myeloid leukemia, as well as, to a lower extent, myelodysplastic syndrome and angioimmunoblastic T-cell lymphoma, and other solid tumors (Dang et al., Annals of Oncology, 27:599-608; Pirozzi and Han, Nature Reviews Clinical Oncology 18:645-661 (2021); Solomou et al., Cancers (Basel). 2023 May 23; 15(11):2883).

### Diagnosis and Prognosis

Methods described herein are used to determine the level of mutant IDH1.R132H sequence in a sample (e.g., a serum sample or a plasma sample or a blood sample) collected from a subject (e.g., a human patient suspected of having a cancer such as glioma). For example, the level of mutant IDH1.R132H sequence can be quantified as IDH1.R132H mutant copies/mL of plasma. The level of mutant IDH1.R132H sequence can then be compared to a reference value to determine whether the subject has or is at risk for a cancer. The reference value can be a control level of mutant IDH1.R132H sequence or a level of wild-type IDH1 sequence, or a ratio thereof. In some embodiments, the control level is a level of mutant IDH1.R132H sequence or wild-type IDH1 sequence in a control sample (e.g., a sample obtained from a healthy subject or population of healthy subjects). As used herein, a healthy subject refers to a subject that is apparently free of a cancer at the time the level of IDH1 is measured or has no history of a cancer.

The control level can also be a predetermined level. Such a predetermined level can represent a level of mutant IDH1.R132H sequence in a population of subjects that do not have or are not at risk for a cancer. The predetermined level can take a variety of forms. For example, it can be a single cut-off value, such as a median or mean. In some embodiments, such a predetermined level can be established based upon comparative groups, such as where one defined group is known to have a cancer and another defined group is known not to have a cancer (e.g., a healthy individual). Alternatively, or in addition to, the predetermined level can be a range including, for example, a range representing the levels of mutant IDH1.R132H sequence and/or wild-type IDH1 sequence in a control population.

The control level as described herein can be determined as described herein and/or by a technology known in the art. In some examples, the control level can be obtained by performing a known method on a control sample as also described herein. In some embodiments, the control level can be obtained from members of a control population (e.g., healthy individuals) and the results can be analyzed by, for example, a computer program, to obtain the control level (a predetermined level) that represents the level of wild-type IDH1 promoter sequence and/or mutant IDH1.R132H sequence in the control population.

By comparing the level of wild-type IDH1 sequence and mutant IDH1.R132H sequence in a sample obtained from a subject to a reference value as described herein, it can be determined as to whether the subject has or is at risk for a cancer (e.g., glioma). For example, if the level of mutant IDH1.R132H sequence in a sample obtained from a candidate subject increased as compared to the reference value (e.g., the level of mutant IDH1.R132H sequence in a sample from a healthy control), the candidate subject might be identified as having or at risk for a cancer. Alternatively, or in addition to, if the level of wild-type IDH1 sequence in a sample obtained from a candidate subject decreased as compared to the reference value (e.g., the level of wild-type IDH1 sequence in a sample from a healthy control), the candidate subject might be identified as having or at risk for a cancer.

As used herein, “an elevated level” or “a level above a reference value” means that the level of mutant IDH1.R132H sequence is higher than a reference value, such as a predetermined threshold or a level of mutant IDH1.R132H sequence in a control sample. An elevated or increased level of mutant IDH1.R132H sequence includes a level of mutant IDH1.R132H sequence that is, for example, 1%, 5%, 10%, 20%, 30%, 40%, 50%, 60%, 70%, 80%, 90%, 100%, 150%, 200%, 300%, 400%, 500% or more above a reference value. An elevated level of the mutant IDH1.R132H sequence also includes increasing a level from a zero state (e.g., no or undetectable mutant IDH1.R132H sequence in a sample) to a non-zero state (e.g., some or detectable mutant IDH1.R132H sequence in the sample).

As used herein, “a decreased level” or “a level below a reference value” means that the level of wild-type IDH1 sequences is lower than a reference value, such as a predetermined threshold or a level of the wild-type IDH1 sequence in a control sample. An reduced or decreased level of the wild-type IDH1 sequence includes a level of wild-type IDH1 sequence that is, for example, 1%, 5%, 10%, 20%, 30%, 40%, 50%, 60%, 70%, 80%, 90%, 100%, 150%, 200%, 300%, 400%, 500% or more below a reference value. A decreased level of the wild-type IDH1 sequence also includes decreasing a level from a non-zero state (e.g., some or detectable wild-type IDH1 sequence in the sample) to a zero state (e.g., no or undetectable mutant IDH1.R132H sequence in a sample).

In some embodiments, the subject is a human patient having, suspected of having, or at risk for having a cancer. A subject might show one or more symptoms of a cancer, e.g., fatigue, a lump or area of thickening that can be felt under the skin, weight changes (e.g., unintended weight loss or weight gain), skin changes (e.g., yellowing, darkening or redness of the skin, sores that cannot heal, or changes to existing moles), changes in bowel or bladder habits, persistent cough or trouble breathing, persistent muscle or joint pain, persistent fevers or night sweats, and unexplained bleeding or bruising.

Alternatively, or in addition to, the subject might show one or more symptoms of a glioma, e.g., headache, memory loss, urinary incontinence, seizures, speech difficulties, confusion, and balance difficulties.

A sample may be obtained from a subject having, suspected of having, or at risk for having a cancer. In some embodiments, the subject has a symptom of a cancer (e.g., glioma) at the time the sample is collected, has no history of a symptom of a cancer, or no history of a cancer. In some embodiments, the subject is resistant to a cancer treatment.

### Evaluation of Cancer Reoccurrence and Treatment Effectiveness

Methods described herein can also be applied to evaluate the reoccurrence of a cancer and/or to evaluate effectiveness of a cancer therapy. For example, multiple samples (e.g., serum or plasma samples) can be collected from a subject to whom a treatment is performed either before and after the treatment or during the course of the treatment.

If the level of mutant IDH1.R132H sequences increases after the treatment or over the course of the treatment (level of mutant IDH1.R132H sequences in a later collected sample as compared to that in an earlier collected sample), remains the same or increases, it indicates that the cancer has reoccurred and/or that the treatment is ineffective.

If the level of mutant IDH1.R132H sequences decreases after the treatment or over the course of the treatment (level of mutant IDH1.R132H sequences in a later collected sample as compared to that in an earlier collected sample), remains the same or decreases, it indicates that the cancer has not reoccurred and/or that the treatment is effective.

Alternatively, or in addition to, if the level of wild-type IDH1 sequences decreases after the treatment or over the course of the treatment (level of wild-type IDH1 sequences in a later collected sample as compared to that in an earlier collected sample), remains the same or decreases, it indicates that the cancer has occurred and/or that the treatment is ineffective.

If the level of wild-type IDH1 sequences increases after the treatment or over the course of the treatment (level of wild-type IDH1 sequences in a later collected sample as compared to that in an earlier collected sample), remains the same or increases, it indicates that the cancer has not reoccurred and/or that the treatment is effective.

If the subject is identified as not responsive to the treatment and/or as having a reoccurrence of the cancer, a higher dose and/or frequency of dosage of the therapeutic agent can be administered to the subject. In some embodiments, the dosage and/or frequency of dosage of the therapy is maintained, lowered, or ceased in a subject identified as responsive to the treatment or not in need of further treatment. Alternatively, a different treatment can be applied to the subject who is found as not responsive to the first treatment and/or who is identified as having reoccurrence of the cancer.

### Non-Clinical Applications

Methods described herein can also be applied to non-clinical uses, e.g., for research purposes. For example, methods described herein can be used to study cancer cell behavior and/or cancer cell mechanisms, which can identify novel biological pathways or processes involved in cancer (e.g., cancer development and/or cancer metastasis).

In some embodiments, methods described herein can be applied to the development of new therapy. For example, the levels of IDH1.R132H mutations can be measured in samples obtained from a subject having been administered anew therapy (e.g., in a clinical trial). In some embodiments, the level of IDH1.R132H mutations can indicate the efficacy of the new therapy or the progress of the cancer in the subject prior to, during, or after the new therapy.

### Kits

Provided here are kits for use in the methods described herein, comprising one or more of the primers described herein, and reagents for performing the methods. For example, the kits can comprise one or more of a forward primer comprising SEQ ID NO: 1; a reverse primer comprising SEQ ID NO:2; a mutant primer comprising SEQ ID NO:4 (or 24); a wild-type primer comprising SEQ ID NO:5 (or 25); and/or a wild-type blocking oligonucleotide comprising SEQ ID NO:8. The wild type blocking oligonucleotide preferably comprises at least one locked nucleic acid (LNA) modification.

In some embodiments, the forward primer comprises a nucleotide sequence that is at least 80% identical, at least 85%, at least 90%, at least 91%, at least 92%, at least 93%, at least 94%, at least 95%, at least 96%, at least 97%, at least 98%, or at least 99% identical to SEQ ID NO: 1. In some embodiments, the forward primer comprises SEQ ID NO: 1. In some embodiments, the forward primer is SEQ ID NO: 1.

In some embodiments, the reverse primer comprises a nucleotide sequence that is at least 80% identical, at least 85%, at least 90%, at least 91%, at least 92%, at least 93%, at least 94%, at least 95%, at least 96%, at least 97%, at least 98%, or at least 99% identical to SEQ ID NO: 2, 18, or 19. In some embodiments, the reverse primer comprises SEQ ID NO: 2, 18, or 19. In some embodiments, the reverse primer is SEQ ID NO: 2.

In some embodiments, the mutant detection primer comprises a nucleotide sequence that is at least 80% identical, at least 85%, at least 90%, at least 91%, at least 92%, at least 93%, at least 94%, at least 95%, at least 96%, at least 97%, at least 98%, or at least 99% identical to SEQ ID NO: 4 or 24. In some embodiments, the mutant detection primer comprises SEQ ID NO: 4 or 24. In some embodiments, the mutant detection primer is SEQ ID NO: 4.

In some embodiments, the wild-type detection primer comprises a nucleotide sequence that is at least 80% identical, at least 85%, at least 90%, at least 91%, at least 92%, at least 93%, at least 94%, at least 95%, at least 96%, at least 97%, at least 98%, or at least 99% identical to SEQ ID NO: 5 or 25. In some embodiments, the wild-type detection primer comprises SEQ ID NO: 5 or 25. In some embodiments, the wild-type detection primer is SEQ ID NO: 5.

In some embodiments, the wild-type blocking oligonucleotide comprises a nucleotide sequence that is at least 80% identical, at least 85%, at least 90%, at least 91%, at least 92%, at least 93%, at least 94%, at least 95%, at least 96%, at least 97%, at least 98%, or at least 99% identical to SEQ ID NO: 8. In some embodiments, the forward primer comprises SEQ ID NO: 8. In some embodiments, the forward primer is SEQ ID NO: 8. Preferably the blocking oligo comprises a 3′ phospho modification.

The detection primers (also referred to herein as probes) can each comprise a fluorophore and a quencher. For example, the mutant detection primers can comprise a first fluorophore and a first quencher, and the second detection primers can comprise a second fluorophore and a second quencher. The first fluorophore and the second fluorophore are different fluorophores, and/or wherein the first quencher and the second quencher are the same quencher. For example, the first fluorophore and the second fluorophore can be FAM, HEX, Cy3, Cy5, and Texas Red, and/or the first quencher and the second quencher can be Iowa Black FQ, Iowa Black RQ, ZEN Quencher, and TAMRA.

### Treatment of a Cancer

A subject identified as having, suspected of having, or at risk for having a cancer (e.g., glioma), as identified using the methods described herein, can be treated with any appropriate therapy. In some embodiments, methods provided herein include administering a cancer therapy to a subject based on the output of the methods described herein, e.g., detecting a IDH1.R132H mutation. Examples of a cancer therapy include, but are not limited to, a chemotherapy, a radiation therapy, a surgical therapy, and an immunotherapy.

In some embodiments, a chemotherapy is administered to the subject. Chemotherapy includes, but is not limited to, alkylating agents (e.g., Cisplatin), nitrosoureas (e.g., Carmustine, Lomustine, Streptozocin), antimetabolites (e.g., Gemcitabine, Hydroxyurea, Methotrexate), anti-tumor antibiotics (e.g., anthracyclines such as Doxorubicin), Topoisomerase inhibitors (e.g., camptothecins, epipodophyllotoxins), mitotic inhibitors (e.g., taxanes, vinca alkaloids), and corticosteroids (e.g., Prednisone, Methylprednisolone, Dexamethasone). In some embodiments, an IDH1 inhibitor is administered, e.g., AG-120 (ivosidenib), AG-881 (vorasidenib), AG-221 (enasidenib), AGI 5198, BAY 1436032, IDH 305, AGI 6780, FT-2102, MFK A, GSK 321, ML 309, and Ds 1001b/AB-218 (Safusidenib) (see, e.g., Solomou et al., Cancers (Basel). 2023 May 23; 15(11):2883).

In some embodiments, a radiation therapy is administered to the subject. Radiation therapy includes, but is not limited to, ionizing radiation, gamma-radiation, neutron beam radiotherapy, electron beam radiotherapy, proton therapy, brachytherapy, and radioactive isotopes and radiosensitizers.

In some embodiments, a surgical therapy is administered to the subject. Surgical therapy includes, but is not limited to, curative surgery (e.g., tumor removal surgery), a preventive surgery, a laparoscopic surgery, and laser surgery.

In some embodiments, an immunotherapy is administered to the subject. Immunotherapy includes, but is not limited to, adoptive cell therapy, cancer vaccine therapy, immune checkpoint inhibitors (e.g., PD-1 inhibitors or PD-L1 inhibitors), oncolytic virus therapy, targeted antibody therapy, and immune-modulating therapy (e.g., cytokine therapy).

Methods described herein comprise administering one type of cancer therapy or multiple types of cancer therapies, which can be referred to as combination therapy. For example, the subject can be treated using a chemotherapy and an immunotherapy. It should be appreciated that any combination of cancer therapies can be administered to the subject. A cancer therapy can be administered one or more times to the subject.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Exemplary Sequences

In some embodiments, the sequence of a protein or nucleic acid used in a composition or method described herein is at least 80%, 85%, 90%, 95%, 97%, 98%, or 99% identical to a sequence set forth herein. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

In some embodiments, the sequence of a protein or nucleic acid used in a composition or method described herein has up to 1, 2, 3, 4, 5, 6, 7, 8, 9, or 10 amino acid substitutions or deletions as compared to a sequence set forth herein. In some embodiments, the substitutions are conservative substitutions.

Human IDH1 RNA-NM_005896-
CDS: 224-1468, Fwd and Reverse primer binding
sites bolded
SEQ ID NO: 26
GGGTTTCTGCAGAGTCTACTTCAGAAGCGGAGGCACTGGGAGTCCGG
TTTGGGATTGCCAGGCTGTGGTTGTGAGTCTGAGCTTGTGAGCGGCT
GTGGCGCCCCAACTCTTCGCCAGCATATCATCCCGGCAGGCGATAAA
CTACATTCAGTTGAGTCTGCAAGACTGGGAGGAACTGGGGTGATAAG
AAATCTATTCACTGTCAAGGTTTATTGAAGTCAAAATGTCCAAAAAA
ATCAGTGGCGGTTCTGTGGTAGAGATGCAAGGAGATGAAATGACACG
AATCATTTGGGAATTGATTAAAGAGAAACTCATTTTTCCCTACGTGG
AATTGGATCTACATAGCTATGATTTAGGCATAGAGAATCGTGATGCC
ACCAACGACCAAGTCACCAAGGATGCTGCAGAAGCTATAAAGAAGCA
TAATGTTGGCGTCAAATGTGCCACTATCACTCCTGATGAGAAGAGGG
TTGAGGAGTTCAAGTTGAAACAAATGTGGAAATCACCAAATGGCACC
ATACGAAATATTCTGGGTGGCACGGTCTTCAGAGAAGCCATTATCTG
CAAAAATATCCCCCGGCTTGTGAGTGGATGGGTAAAACCTATCATCA
TAGGTCGTCATGCTTATGGGGATCAATACAGAGCAACTGATTTTGTT
GTTCCTGGGCCTGGAAAAGTAGAGATAACCTACACACCAAGTGACGG
AACCCAAAAGGTGACATACCTGGTACATAACTTTGAAGAAGGTGGTG
GTGTTGCCATGGGGATGTATAATCAAGATAAGTCAATTGAAGATTTT
GCACACAGTTCCTTCCAAATGGCTCTGTCTAAGGGTTGGCCTTTGTA
TCTGAGCACCAAAAACACTATTCTGAAGAAATATGATGGGCGTTTTA
AAGACATCTTTCAGGAGATATATGACAAGCAGTACAAGTCCCAGTTT
GAAGCTCAAAAGATCTGGTATGAGCATAGGCTCATCGACGACATGGT
GGCCCAAGCTATGAAATCAGAGGGAGGCTTCATCTGGGCCTGTAAAA
ACTATGATGGTGACGTGCAGTCGGACTCTGTGGCCCAAGGGTATGGC
TCTCTCGGCATGATGACCAGCGTGCTGGTTTGTCCAGATGGCAAGAC
AGTAGAAGCAGAGGCTGCCCACGGGACTGTAACCCGTCACTACCGCA
TGTACCAGAAAGGACAGGAGACGTCCACCAATCCCATTGCTTCCATT
TTTGCCTGGACCAGAGGGTTAGCCCACAGAGCAAAGCTTGATAACAA
TAAAGAGCTTGCCTTCTTTGCAAATGCTTTGGAAGAAGTCTCTATTG
AGACAATTGAGGCTGGCTTCATGACCAAGGACTTGGCTGCTTGCATT
AAAGGTTTACCCAATGTGCAACGTTCTGACTACTTGAATACATTTGA
GTTCATGGATAAACTTGGAGAAAACTTGAAGATCAAACTAGCTCAGG
CCAAACTTTAAGTTCATACCTGAGCTAAGAAGGATAATTGTCTTTTG
GTAACTAGGTCTACAGGTTTACATTTTTCTGTGTTACACTCAAGGAT
AAAGGCAAAATCAATTTTGTAATTTGTTTAGAAGCCAGAGTTTATCT
TTTCTATAAGTTTACAGCCTTTTTCTTATATATACAGTTATTGCCAC
CTTTGTGAACATGGCAAGGGACTTTTTTACAATTTTTATTTTATTTT
CTAGTACCAGCCTAGGAATTCGGTTAGTACTCATTTGTATTCACTGT
CACTTTTTCTCATGTTCTAATTATAAATGACCAAAATCAAGATTGCT
CAAAAGGGTAAATGATAGCCACAGTATTGCTCCCTAAAATATGCATA
AAGTAGAAATTCACTGCCTTCCCCTCCTGTCCATGACCTTGGGCACA
GGGAAGTTCTGGTGTCATAGATATCCCGTTTTGTGAGGTAGAGCTGT
GCATTAAACTTGCACATGACTGGAACGAAGTAGGAGTGCAACTCAAA
TGTGTTGAAGATACTGCAGTCATTTTTGTAAAGACCTTGCTGAATGT
TTCCAATAGACTAAATACTGTTTAGGCCGCAGGAGAGTTTGGAATCC
GGAATAAATACTACCTGGAGGTTTGTCCTCTCCATTTTTCTCTTTCT
CCTCCTGGCCTGGCCTGAATATTATACTACTCTAAATAGCATATTTC
ATCCAAGTGCAATAATGTAAGCTGAATCTTTTTTGGACTTCTGCTGG
CCTGTTTTATTTCTTTTATATAAATGTGATTTCTCAGAAATTGATAT
TAAACACTATCTTATCTTCTCCTGAACTGTTGATTTTAATTAAAATT
AAGTGCTAATTACCA

### EXAMPLES

The invention is further described in the following examples, which do not limit the scope of the invention described in the claims.

### Methods

The following methods and materials were used in the Examples set forth below, unless otherwise noted.

### Study Population

Disease group participants recruited from the clinics to our biobank were not compensated for participation. Healthy control participants recruited from the general public were offered a monetary compensation for each blood draw they complete. The study population (N=133) consisted of IDH1.R132H mutant; n=80, IDH1 wild-type; n=44 and age matched healthy controls; n=9). Brain tumor patients aged 18 years or older underwent surgery at the Massachusetts General Hospital (MGH) for biopsy or resection of a primary tumor lesion. Gender and/or sex was not considered in the study design. The gender and/or sex of all participants was determined based on self-report. Clinical IDH.R132H status was established with either immunohistochemistry (IHC), Rapid IDH1 Analysis (DNA based qPCR), or by the MGH SNaPShot Panel. Additional inclusion criteria for the study population included histopathological confirmation of disease. Healthy control participants with a history of oncologic, neurologic, or ongoing infectious conditions were excluded from the study. The study was performed on samples collected between 2014 and 2024. All IDH1.R132H samples were included from the biobank and matched with IDH-wild-type controls. Patient demographics and clinical parameters are depicted in Table 1A and Table 1B.

TABLE 1A
IDH1.R132H mutant study population demographics and disease parameters.

| | | | |
|---|---|---|---|
| | Cohort 1, (N = 10) | Cohort 2, (N = 9) | Cohort 3, (N = 61) |
| Parameter | n (%) | n (%) | n (%) |

Age (yr)

| | | | | | | |
|---|---|---|---|---|---|---|
| ≤40 | 5 | (50.0) | 1 | (11.1) | 30 | (49.1) |
| >40 | 5 | (50.0) | 8 | (88.9) | 31 | (50.8) |

Gender

| | | | | | | |
|---|---|---|---|---|---|---|
| Male | 6 | (60.0) | 6 | (66.7) | 38 | (62.3) |
| Female | 4 | (40.0) | 3 | (33.3) | 23 | (37.7) |

WHO Grade

| | | | | | | |
|---|---|---|---|---|---|---|
| 2 | 5 | (50.0) | 2 | (22.2) | 17 | (27.9) |
| 3 | 5 | (50.0) | 6 | (66.7) | 31 | (50.8) |
| 4 | 0 | (0.0) | 1 | (11.1) | 13 | (21.3) |

Diagnosis

| Astrocytoma, Grade 2-3 | 9 | (90.0) | 6 | (66.7) | 32 | (52.5) |
|---|---|---|---|---|---|---|
| Oligodendroglioma, Grade | 1 | (10.0) | 2 | (22.2) | 16 | (26.2) |
| 2-3 | | | | | | |
| Astrocytoma, Grade 4 | 0 | (0.0) | 1 | (11.1) | 13 | (21.3) |

Cortical Location

| | | | | | | |
|---|---|---|---|---|---|---|
| Frontal | 2 | (20.0) | 3 | (33.3) | 41 | (67.2) |
| Parietal | 2 | (20.0) | 2 | (22.2) | 4 | (6.6) |
| Temporal | 5 | (50.0) | 3 | (33.3) | 11 | (18.0) |
| Other | 1 | (10.0) | 1 | (11.1) | 5 | (8.2) |

Cerebral Hemisphere

| | | | | | | |
|---|---|---|---|---|---|---|
| Dominant | 6 | (60.0) | 5 | (55.6) | 34 | (55.7) |
| Non-dominant | 4 | (40.0) | 4 | (44.4) | 24 | (39.3) |

Tumor Volume (cm³)

| | | | | | | |
|---|---|---|---|---|---|---|
| <20 | 2 | (20.0) | 0 | (0.0) | 15 | (24.6) |
| 20-50 | 3 | (30.0) | 2 | (22.2) | 15 | (24.6) |
| 51-100 | 2 | (20.0) | 3 | (33.3) | 12 | (19.7) |
| >100 | 2 | (20.0) | 3 | (33.3) | 13 | (21.3) |
| ill-defined | 1 | (10.0) | 1 | (11.1) | 5 | (8.2) |

Recurrence

| | | | | | | |
|---|---|---|---|---|---|---|
| Yes | 3 | (30.0) | 5 | (55.6) | 28 | (45.9) |
| No | 7 | (70.0) | 4 | (44.4) | 31 | (50.1) |

Post-Operative Treatment

| | | | | | | |
|---|---|---|---|---|---|---|
| RT only | 5 | (50.0) | 5 | (55.6) | 41 | (67.2) |
| RT + TMZ | 2 | (20.0) | 1 | (11.1) | 11 | (18.0) |
| Clinical Trial Participant | 3 | (30.0) | 1 | (11.1) | 4 | (6.6) |
| Surveillance | 1 | (10.0) | 3 | (33.3) | 9 | (14.8) |

MGMT status

| | | | | | | |
|---|---|---|---|---|---|---|
| methylated | 4 | (40.0) | 3 | (33.3) | 20 | (32.8) |
| unmethylated | 3 | (30.0) | 1 | (11.1) | 17 | (28.9) |

TP53 status

| | | | | | | |
|---|---|---|---|---|---|---|
| wild-type | 1 | (10.0) | 3 | (33.3) | 10 | (16.4) |
| mutant | 7 | (70.0) | 6 | (66.7) | 48 | (78.7) |

ATRX status

| | | | | | | |
|---|---|---|---|---|---|---|
| wild-type | 1 | (10.0) | 4 | (44.4) | 21 | (34.4) |
| mutant | 7 | (70.0) | 5 | (55.6) | 36 | (59.0) |

CDKN2A status

| | | | | | | |
|---|---|---|---|---|---|---|
| wild-type | 4 | (40.0) | 5 | (55.6) | 28 | (45.9) |
| mutant | 0 | (0.0) | 0 | (0.0) | 8 | (13.1) |

Abbreviations: WHO; World Health Organization, RT; Radiation therapy, TMZ; Temozolamide, MGMT; O⁶-methylguanine-DNA methyl-transferase, TP53; Tumor Protein 53, ATRX; α-thalassemia/mental-retardation-syndrome-X-linked gene, CDKN2A; cyclin-dependent kinase inhibitor 2A.

TABLE 1B
IDH1 wild-type study population demographics and disease parameters.

| | | | |
|---|---|---|---|
| | Cohort 1, (N = 11) | Cohort 2, (N = 11) | Cohort 3, (N = 34) |
| Parameter | n (%) | n (%) | n (%) |

Category

| | | | | | | |
|---|---|---|---|---|---|---|
| Disease | 11 | (100.0) | 7 | (63.4) | 29 | (85.3) |
| Healthy | 0 | (0.0) | 4 | (36.4) | 5 | (14.7) |

Age (yr)

| | | | | | | |
|---|---|---|---|---|---|---|
| ≤60 | 7 | (63.6) | 6 | (54.5) | 20 | (58.8) |
| >60 | 4 | (36.4) | 5 | (45.5) | 14 | (41.2) |

Gender

| | | | | | | |
|---|---|---|---|---|---|---|
| Male | 8 | (72.7) | 8 | (72.7) | 25 | (73.5) |
| Female | 3 | (27.3) | 3 | (27.3) | 9 | (26.5) |

WHO Grade

| | | | | | | |
|---|---|---|---|---|---|---|
| 2 | 2 | (18.2) | 0 | (0.0) | 3 | (10.3) |
| 3 | 0 | (0.0) | 0 | (0.0) | 0 | (0.0) |
| 4 | 9 | (81.8) | 7 | (100.0) | 26 | (89.7) |

Diagnosis

| | | | | | | |
|---|---|---|---|---|---|---|
| Astrocytoma | 3 | (27.3) | 1 | (14.3) | 6 | (20.7) |
| Primary GBM | 8 | (72.7) | 5 | (71.4) | 21 | (72.4) |
| Secondary GBM | 0 | (0.0) | 1 | (14.3) | 1 | (3.4) |

Cortical Location

| | | | | | | |
|---|---|---|---|---|---|---|
| Frontal | 2 | (18.2) | 1 | (14.3) | 9 | (31.0) |
| Parietal | 2 | (18.2) | 5 | (71.4) | 6 | (20.7) |
| Temporal | 6 | (54.5) | 1 | (14.3) | 9 | (31.0) |
| Other | 1 | (9.1) | 0 | (0.0) | 5 | (17.2) |

Cerebral Hemisphere

| | | | | | | |
|---|---|---|---|---|---|---|
| Dominant | 5 | (45.5) | 0 | (0.0) | 15 | (51.7) |
| Non-dominant | 6 | (54.5) | 7 | (100.0) | 11 | (37.9) |

Tumor Volume (cm³)

| | | | | | | |
|---|---|---|---|---|---|---|
| ≤50 | 5 | (45.5) | 3 | (42.9) | 23 | (79.3) |
| 51 - 100 | 1 | (9.1) | 1 | (14.3) | 3 | (10.3) |
| >100 | 2 | (18.2) | 2 | (28.6) | 0 | (0.0) |
| ill-defined | 2 | (18.2) | 1 | (14.3) | 3 | (10.3) |

Post-Operative Treatment

| | | | | | | |
|---|---|---|---|---|---|---|
| RT + TMZ | 11 | (100) | 6 | (85.7) | 20 | (69.0) |
| Clinical Trial Participant | 0 | (0) | 0 | (0) | 4 | (13.8) |
| Surveillance | 5 | (45.5) | 3 | (42.9) | 9 | (31.0) |

TERTp (C228T, C250T) status

| | | | | | | |
|---|---|---|---|---|---|---|
| wild-type | 1 | (9.1) | 6 | (85.7) | 6 | (20.7) |
| mutant | 5 | (45.5) | 1 | (14.3) | 13 | (44.8) |

MGMT status

| | | | | | | |
|---|---|---|---|---|---|---|
| methylated | 2 | (18.2) | 3 | (42.9) | 10 | (34.5) |
| unmethylated | 7 | (63.6) | 4 | (57.1) | 17 | (58.6) |

TP53 status

| | | | | | | |
|---|---|---|---|---|---|---|
| wild-type | 3 | (27.3) | 1 | (14.3) | 7 | (24.1) |
| mutant | 3 | (27.3) | 4 | (57.1) | 11 | (37.9) |

CDKN2A status

| | | | | | | |
|---|---|---|---|---|---|---|
| wild-type | 3 | (27.3) | 6 | (85.7) | 13 | (44.8) |
| mutant | 3 | (27.3) | 1 | (14.3) | 5 | (17.2) |

Abbreviations: WHO; World Health Organization, GBM; Glioblastoma, RT; Radiation therapy, TMZ; Temozolamide, TERTp; Telomerase reverse transcriptase promoter, MGMT; O6-methylguanine-DNA methyl-transferase, TP53; Tumor Protein 53, CDKN2A; cyclin-dependent kinase inhibitor 2A.

### Tumor Tissue Processing

Tumor tissue was micro dissected and suspended in RNALATER storage reagent (Ambion) or flash-frozen and stored at −80° C.

### Patient Plasma Processing

Whole blood was collected using K(2 EDTA tubes with an inert gel barrier (BD Vacutainer Blood Collection Tubes), from pre-operatively placed arterial lines or venipuncture. Within 2 hours of collection, samples were centrifuged at 1,100×g for 10 minutes at 20° C. to separate the plasma from the hematocrit and filtered using 0.8 μM filters. 1 ml aliquots were stored at −80° C. for later downstream analysis. With the exception of the longitudinal samples, all baseline samples were collected prior to surgical resection.

RNA and DNA Isolation from Tumor Tissue

Frozen tumor tissue was thawed and lysed in 1-2 ml of ice-cold TRIZOL Reagent, a solution of phenol and guanidine isothiocyanate (ThermoFisher). Lysate was homogenized by passing through a 20-gauge RNAse-free needle 10 times. Total RNA was then extracted as per the manufacturer's protocol and eluted in 20 μl of nuclease free water (Invitrogen). All RNA samples (cell lines, tumor tissue) were assessed for purity with the NanoDrop One spectrophotometer (ThermoFisher, Waltham, MA, USA). Agilent RNA 6000 Pico kit for analysis of RNA fragments was used with Agilent Technologies 2100 Bioanalyzer (Waldbronn, Germany) to determine the concentration and RIN (RNA Integrity Number) value of the samples. Isolated RNA was stored at −80° C. prior to downstream analysis.

Co-Isolation of DNA and RNA from Tumor Tissue

Frozen tumor tissue was thawed on ice. For each sample, DNA and RNA extraction was done in parallel using AllPrep DNA/RNA/miRNA Universal Kit (QIAgen), per manufacturer's recommendations. DNA was eluted in 100 μl of the elution buffer (QIAgen) and stored at −20° C. until further processing. RNA was eluted in 20 μl of nuclease-free water (QIAgen) and stored at −80° C. until further processing.

EV RNA Extraction from Plasma Samples

In order to isolate the analyte of interest, 2 ml plasma was obtained from each clinical sample (IDH.R132H mut, IDH wild-type, Healthy) and thawed on ice. Extracellular vesicle RNA (EV RNA) was isolated using the EXOLUTION kit, per manufacturer's recommendations. EV RNA was eluted in 17 μl of nuclease free water (Qiagen) and assessed for concentration and purity with the NanoDrop One spectrophotometer (ThermoFisher Scientific).

Co-Isolation of EV RNA, EV DNA, and cfDNA from Plasma Samples

In order to isolate the analyte of interest, 2 ml plasma was obtained from each clinical sample (IDH.R132H mut, IDH wild-type, Healthy) and thawed on ice. EXOLUTIONPLUS kit was utilized for one step isolation of EV RNA, EV DNA, and cell free DNA (cfDNA). Isolated multianalyte was eluted in 17 μl of nuclease free water (Qiagen) and assessed for concentration and purity with the NanoDrop One spectrophotometer (ThermoFisher Scientific).

### Reverse Transcription

Messenger RNA (mRNA) isolated from tumor tissue and cell lines, and EV RNA isolated from plasma samples, was reverse transcribed into cDNA using the SuperScript™ VILO™ cDNA Synthesis Kit (ThermoFisher Scientific). The protocol was performed as per manufacturer's instructions. Briefly, the 20 μl reaction volume was prepared using 4 μl of 5×VILO™ Reaction Mix, 2 μl of 10×SuperScript™ Enzyme Mix, RNA (tumor tissue: 1 μl, Plasma (EV RNA): 16 μl), and DEPC-treated water. The thermocycling conditions were as follows: 25° C. for 10 min, 42° C. for 60 min, and termination at 85° C. for 5 min. The resulting cDNA was used immediately or stored at −20° C. for later use.

### Primers and Probes

Through the course of optimization and preliminary testing, at least six different primer pairs specific to IDH.R132H were designed and tested. The primer sets generated amplicons of varying lengths, ranging from 49 bp to 89 bp. The key criteria in selecting the optimal primer set included; target amplicon size<100 bp, minimum formation of primer dimers, and absence of non-specific products. Tools used to design and assess different parameters include Primer-BLAST, Serial Cloner and Primer3Plus. Different primer sets were also compared on the basis of GC content, Tₘ(° C.) mismatch, self-dimerization and hairpin formation; a number were tested, see Table 2. The final primer set selected for further optimization produced a target amplicon of 61 bp. Sequence for primers and probe are as follows: Forward mRNA primer (5′-AGTGGATGGGTAAAACC-3′ (SEQ ID NO:1)), Reverse mRNA primer (complementary to 5′-GATCAATACAGAGCAAC-3′ (SEQ ID NO:3), e.g., 5′-GTTGCTCTGTATTGATC-3′ (SEQ ID NO:2), modified IDH mutant probe (5′-/FAM/TCATAG+GTC+ATC+ATG+CTTA/IABkFQ/-3′(SEQ ID NO:4)), and modified IDH Wild-type probe (5′-/HEX/TCATAG+GTC+GTC+ATG+CTTA/IABkFQ/-3′ (SEQ ID NO:5)). All oligonucleotide preparations were synthesized by ThermoFisher Scientific and Integrated DNA Technologies. Primers were purified by desalting and resuspended in DEPC-treated water, at a final concentration of 100 μM. Mutant and Wild-type probes were HPLC purified and resuspended in 1×TE 100 pmol/μl.

TABLE 2
Probe and Primer Sequences

| | | SEQ |
|---|---|---|
| Primer/ | | ID |
| Probe name | Sequence | NO: |
| Fwd49 | AGTGGATGGGTAAAACC | 1 |
| Rev49 | GATCCCCATAAGCATGA | 11 |
| Rev 62 | GTTGCTCTGTATTGATC | 2 |
| Probe 1 | TATCATCATAGGTCA | 12 |
| Probe 2 | TATCATCATAGGTCAT | 13 |
| Probe 3 | TATCATCATAGGTCATC | 14 |
| Probe 4 | TCATAGGTCATCATGCTTA | 15 |
| Fwd101 | ATCTGCAAAAATATCCCCCGGC | 16 |
| Rev49A | GATCCCCATAAGGATGA | 17 |
| Rev63A | AGTTGCTCTGTATTGAT | 18 |
| Rev63b | AGTTGCTCTGTATTGATCC | 19 |
| Rev101 | ACAACAAAATCAGTTGCTCTGTA | 20 |
| IDH mutant probe 1 | /FAM/TCATAG+GTC+ATC+AT | 4 |
| | G+CTTA/IABkFQ/ | |
| IDH mutant probe 1 | TCATAGGTCATCATGCTTA | 24 |
| unmodified | | |
| sequence | | |
| IDH mutant probe 2 | ATCATCATAGGTCAT | 21 |
| IDH mutant probe 3 | CATCATAGGTCATC | 22 |
| IDH WT probe 1 | /HEX/TCATAG+GTC+GTC+AT | 5 |
| | G+CTTA/IABkFQ/ | |
| IDH WT probe 1 | TCATAGGTCGTCATGCTTA | 25 |
| unmodified | | |
| sequence | | |
| IDH WT probe 2 | ATCATCATAGGTCG | 23 |

FAM: 5′ FAM fluorophore
+: LNA modification
IABkFQ: IOWA BLACK FQ quencher
HEX: 5′HEX fluorophore

### Wild-Type Blocker

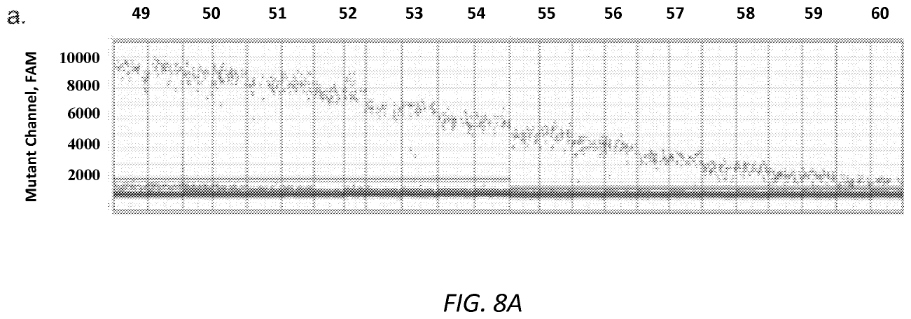

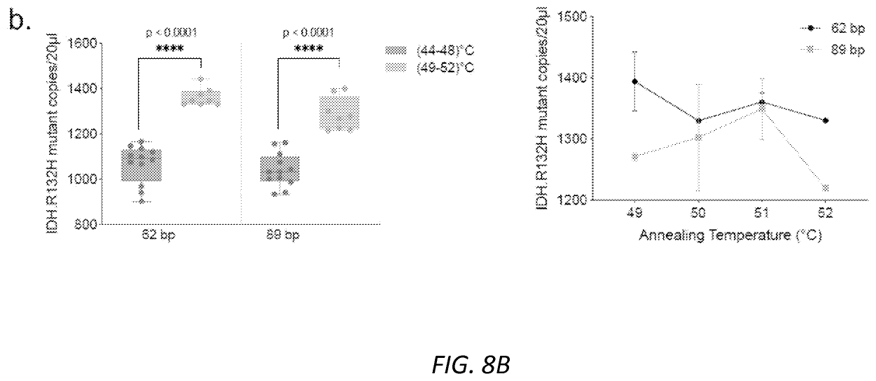

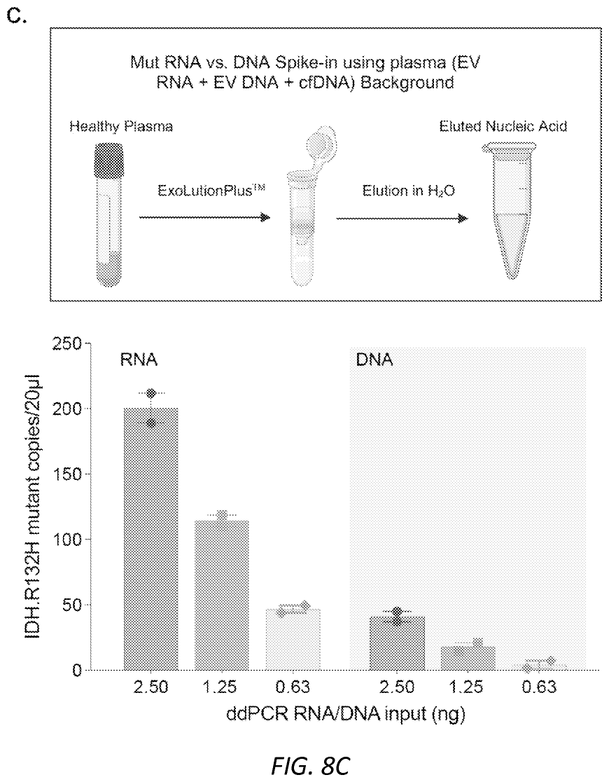

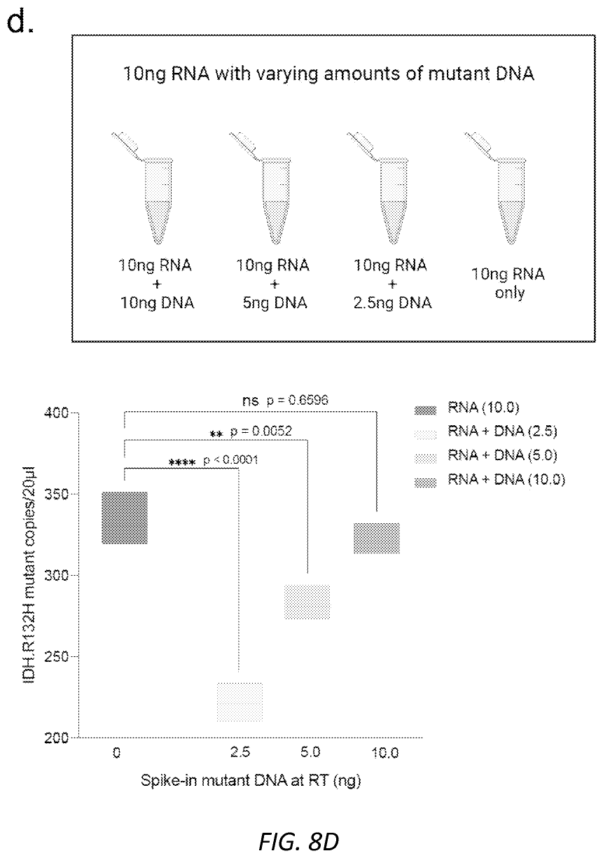

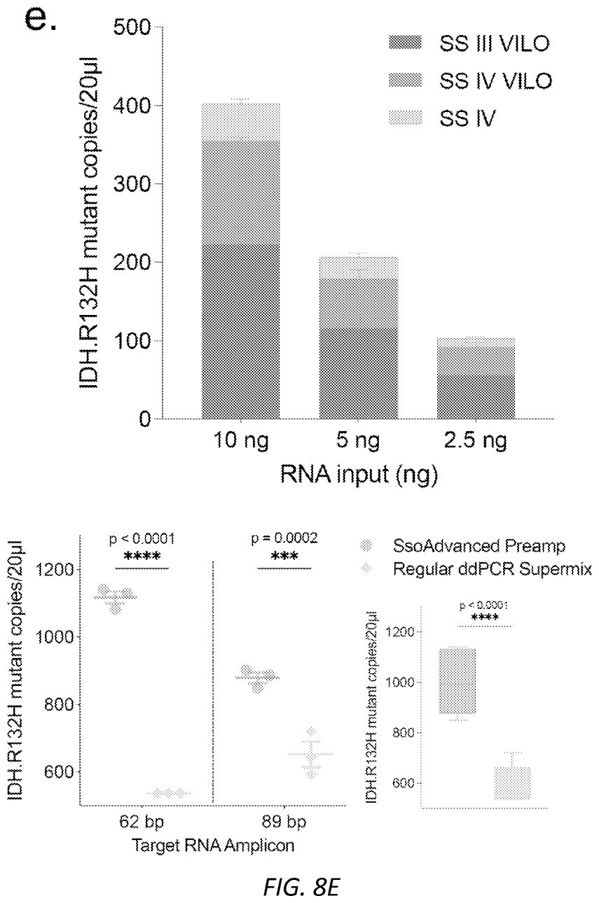

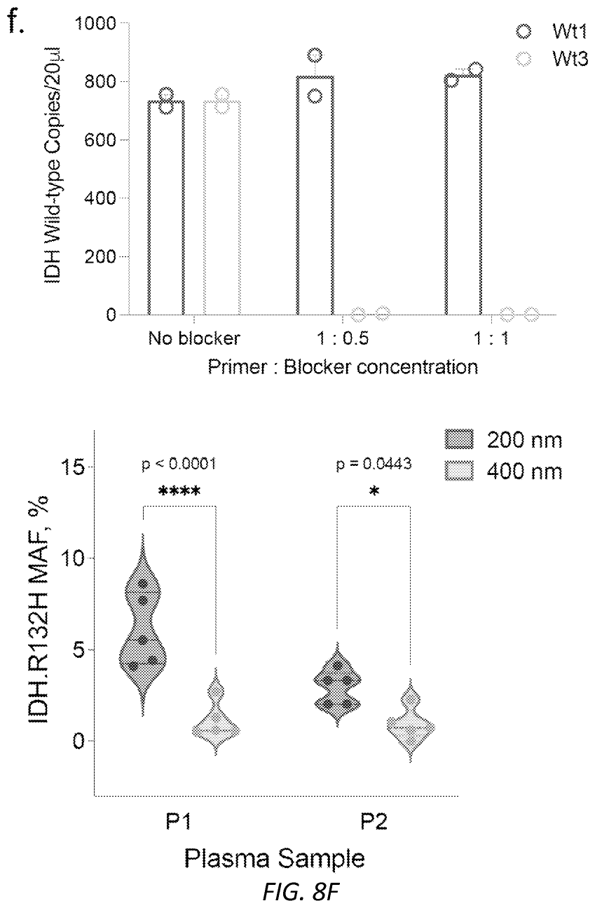

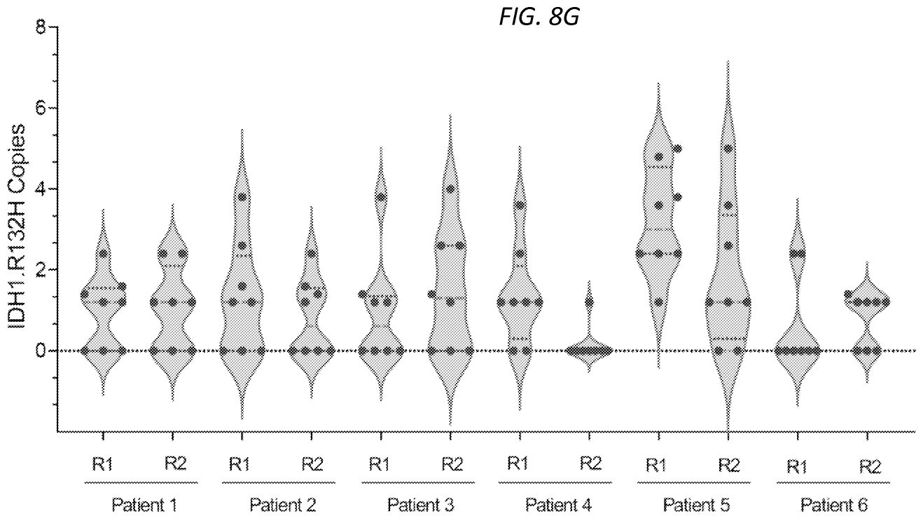

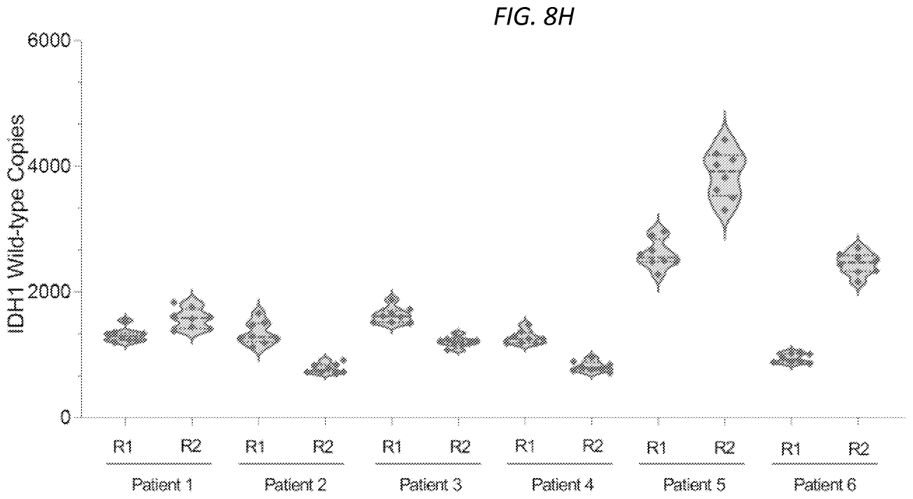

To facilitate minimization of wild-type signal amplification during preamplification step, three different wild-type blockers were designed and tested. All oligonucleotide preparations were synthesized by Integrated DNA Technologies and resuspended in DEPC-treated water, at a final concentration of 100 μM. The wild-type blockers were designed with selected 3′-phosphate modifications and the sequences are as follows: Wt1 (5′-CATAGGTCTCATGCTTA/3Phos/-3′ (SEQ ID NO: 6)), Wt2 (5′-CCCCATAAGCATGAGACCTATG/3Phos/-3′ (SEQ ID NO: 7)), Wt3 (5′-CCCCATAAGCATGACGACCTATG/3Phos/-3′ (SEQ ID NO: 8)). As per initial testing, Wt1 and Wt3 blockers were selected for further testing (FIG. 8f). Following optimization in tumor tissue and plasma, Wt3 sequence was selected as the wild-type blocker.

Optimized Droplet Digital PCR (ddPCR) Assay in Tumor Tissue and Cell Lines Using Bio-Rad System

Droplet digital PCR (ddPCR) was performed using purified cDNA obtained from reverse transcription of RNA isolated from cell lines and tumor tissue. The ddPCR reaction was performed in the Applied Biosystems™ 96-well Thermal Cycler (ThermoFisher Scientific) in a final volume of 20 μl. The optimized reaction mixture was prepared using 10 μl of ddPCR Supermix for Probes (no dUTP) (Bio-Rad), 1000 nM forward and reverse primers and 250 nM mutant and wild-type probe, cDNA, and water. The prepared reaction mixture was transferred to the wells of DG8 cartridge. To generate the droplets, 65 μl of Droplet generation oil for probes (Bio-Rad) was added and the plate was loaded into the QX200 Droplet generator (Bio-Rad). The droplet emulsions were then carefully transferred to a semi-skirted, PCR-clean 96 well plate (Eppendorf) using a multichannel pipette. Lastly, the plate was sealed using PX1 PCR plate sealer (Bio-Rad). Based on the annealing temperature gradient optimization experiment, the final thermal cycling conditions were as follows: initial enzyme activation at 95° C. (510% ramp) for 10 min, then 40 cycles of denaturation at 94° C. (51% ramp) for 30 s, and annealing/extension at 51.0° C. for 1 min, followed by enzyme inactivation at 98° C. for 10 min and final hold at 4° C. until analysis. Droplets were analyzed using QX 200 droplet reader (Bio-Rad) and data were acquired and analyzed with QuantaSoft analysis software (Bio-Rad).

Optimized Droplet Digital PCR (ddPCR) Assay in Plasma Derived Extracellular Vesicles (EVs) Using Bio-Rad System

Droplet digital PCR (ddPCR) was performed using purified cDNA obtained from reverse transcription of EV RNA isolated from plasma samples. For plasma samples, detection of low levels of mutant EV RNA was enhanced via PCR pre-amplification. The pre-amplification ddPCR was performed in the Applied Biosystems™ 96-well Thermal Cycler (ThermoFisher Scientific) in a final volume of 20 μl. The optimized reaction mixture was prepared using 6.5 μl of cDNA, 10 μl of SSOADVANCED™ PreAmp Supermix (Bio-Rad), 100 nM forward and reverse primers, 200 nM wild-type blocker, and water. Each plasma sample cDNA was preamplified in triplicates (three preamp well, 20 μl reactions). The thermocycling conditions were as follows: initial enzyme activation at 95° C. (51% ramp) for 10 min, then 10 cycles of denaturation at 94° C. (51% ramp) for 30 s, and annealing/extension at 51.0° C. for 1 min, followed by enzyme inactivation at 98° C. for 10 min. Following preamplification, the replicates from each plasma sample were pooled. Preamplified product was then diluted by adding 31 μl of water to 1 μl preamplified product. The final ddPCR was performed using the diluted pre-amplified product in a final reaction volume of 20 μl. The reaction mixture was prepared using 2 μl of diluted pre-amplified product, 10 μl of ddPCR Supermix for Probes (no dUTP) (Bio-Rad), 1000 nM forward and reverse primers and 250 nM mutant and wild-type probe. Each individual sample was run in eight replicates. The final thermal cycling conditions were as follows: initial enzyme activation at 95° C. (51% ramp) for 10 min, then 40 cycles of denaturation at 94° C. (51% ramp) for 30 s, and annealing/extension at 51.0° C. for 1 min, followed by enzyme inactivation at 98° C. for 10 min and final hold at 4° C. until analysis. Droplets were analyzed using QX 200 droplet reader (Bio-Rad) and data were acquired and analyzed with QuantaSoft analysis software (Bio-Rad).

Optimized Droplet Digital PCR (ddPCR) Assay Using the QIACUITY System

The final dPCR was performed using the diluted pre-amplified product in a final reaction volume of 40 μl. The reaction mixture was prepared using 2 μl of diluted pre-amplified product, 10 μl of 4×QIAcuity Probe PCR Mastermix (Qiagen), 1000 nM forward and reverse primers and 250 nM mutant and wild-type probes. Each individual sample was run in eight replicates. The sample reaction mixtures were transferred to 26K 24-well Qiacuity Nanoplates (Qiagen) and sealed with Qiacuity Nanoplate rubber seals (Qiagen).

The sealed final dPCR plate was placed in the Qiacuity One 5-plex instrument under the following thermocycling conditions: initial enzyme activation at 95° C. for 2 minutes, 40 cycles of denaturation at 95° C. for 15 seconds, and annealing/extension at 55° C. for 30 seconds. Droplets were analyzed and data were acquired using QIAcuity Software Suite (Qiagen).

Droplet Digital PCR (ddPCR) dMIQE 2020 Guideline Compliance

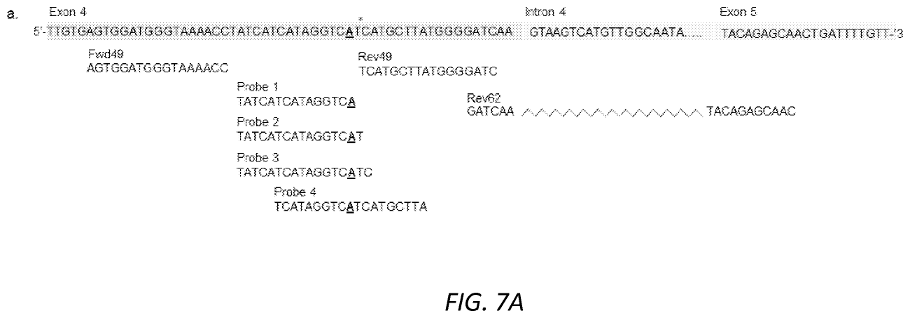

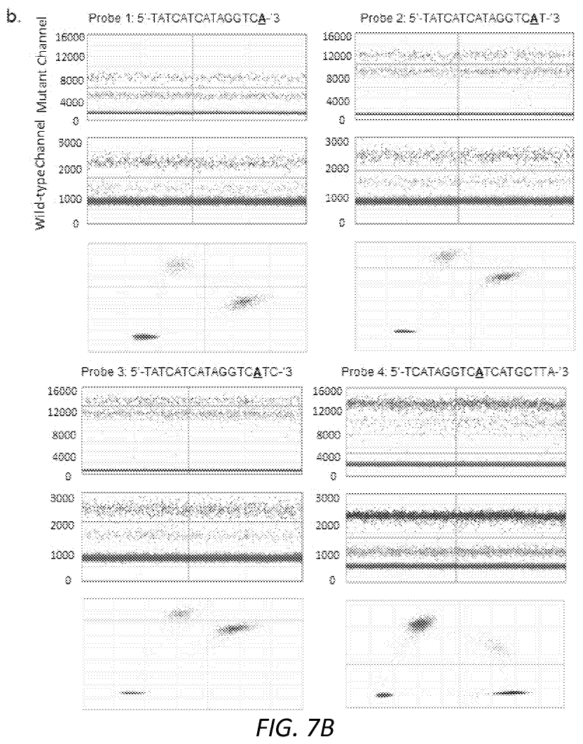

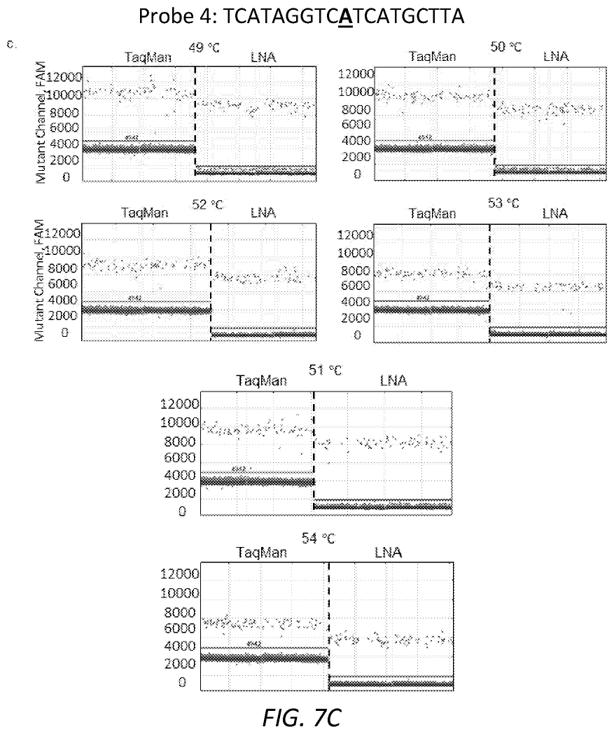

This section of the methods describes our compliance with the updated 2020 dMIQE Guidelines for the technical development of a plasma-based ddPCR assay for the IDH.R132H mutation detection. Specimen type numbers, sampling procedure, aliquotation, conditions and duration are provided in the following sections of the Methods: Study Population, Tumor Tissue Processing, Cell Lines, and Plasma Processing. Details about the specific Extraction techniques, Nucleic Acid Assessment/Storage, and Reverse Transcription are provided in sections: RNA Isolation from cell lines and tumor tissue, and Exosomal RNA Isolation from plasma samples. Information about the ddPCR Oligonucleotides and its target sequences are provided along with the protocol both in FIG. 7 and in the section entitled Primers and Probe. Details about the assay analytical validation are provided in FIG. 1. Finally, information regarding data analysis is provided both in the Results and Methods (see below).

### Quantification of IDH.R132H Mutation in Plasma

The number of IDH1.R132H copies per mL of plasma was calculated from QuantaSoft data as follows: Copies/ml plasma=C EV/TV/P where C=copies per 20 μL, EV=exNA elution volume (μl), TV=exNA input into ddPCR reaction (μl) and p=plasma volume (ml). Only samples with >10,000 droplets/well were included in the analysis. Eight wells are analyzed in parallel for each sample. A positive sample is considered when at least three out of eight replicates have a signal. Number of IDH1 wild-type copies per ml of plasma was calculated using the same formula. We report IDH1.R132H Mutant Allele Frequency (MAF, %) calculated as follows: IDH1.R132H copies/(IDH1.R132H copies+IDH1 wild-type copies)×100%.

### Statistical Analysis

Statistical analysis was performed using the two-tailed Student's t-test (2 groups) and Ordinary one-way ANOVA (Analysis of Variance, >2 groups) in GraphPad Prism Version 10.1.1 software and p<0.05 was considered to be statistically significant. The results are presented as Mean±SEM. For normalization of longitudinal monitoring data (FIG. 6d), the statistical normalization was performed using GraphPad Prism. Briefly, it normalizes data between 0 and 100%, where zero is defined as the smallest value (MAF %) in each data set and one hundred is defined as the largest mean (MAF %). Data from each patient are normalized to a value in (0,1) so that the minimum within the patient takes value 0, the maximum takes value 1, and the value in between is normalized to the proportion between the minimum and the maximum. The mathematical formula for the normalization is: Normalized (x)=(x−min)/(max−min), where min and max are the minimum and maximum within the patient, respectively.

### Data Visualization

FIG. 2, panels a-b were created with BioRender.com.

### Institutional Review Board Statement

Our studies were conducted in accordance with principles for human experimentation as defined in the U.S. Common Rule and were approved by the Human Investigational Review Board of each study center under Partners institutional review board (IRB)-approved protocol number 2017P001581. All healthy control subjects were screened for pertinent oncologic and neurologic medical histories. Individuals with a history of cancer, neurological disorders, and infectious diseases were excluded from the study.

### Informed Consent Statement

All samples were collected with written informed consent after the patient was advised of the potential risks and benefits, as well as the investigational nature of the study.

### Example 1. Detection of IDH1.R132H Mutation Via RNA Yields Higher Sensitivity Compared to DNA-Based Methods

The canonical alteration in IDH1 mutant glioma, IDH1.R132H (IDH1mt) mutation occurs as a result of G>A transition at nucleotide position 395, in the tail region of exon 4 with close proximity to the exon 5 nucleotide region (FIG. 7a). This unique location allows possible variant detection in both DNA and RNA. To investigate the contribution of IDH1mt signal in both RNA and DNA in different sample types, we designed and optimized both DNA (amplicon size, 49 bp) and RNA (amplicon size, 62 bp) based assays (FIG. 7a; Table 2). Four different combinations of mutant and wild-type probes were tested to achieve optimal signal separation in 1-D and 2-D amplitude plots with minimal cross reactivity of the two probes (FIG. 7b). The final assays were then utilized for direct DNA vs RNA IDH1mt signal comparison.

First, we extracted DNA and RNA from IDH1.R132H mutant tumor tissue of one patient (described in Methods). Both analytes were diluted to yield a wide range of DNA/RNA inputs (30.0-0.3 ng; FIG. 1a). IDH1.R132H mutant allele frequency (MAF, %) measured across all concentrations was significantly (p<0.0001, ****) higher in RNA (34.40%, 95% CI; 32.69%-36.11%) as compared to DNA (12.31%, 95% CI; 11.09%-13.53%) from the same specimen (FIG. 1a). Next, we compared the RNA/DNA enrichment ratio in a tumor tissue cohort (n=23) of different IDH1mt glioma subtypes; Astrocytoma, Grade 2-3 (n=12), Oligodendroglioma, Grade 2-3 (n=7), and Astrocytoma, Grade 4 (n=4) (FIG. 1b). We again observed a significantly higher enrichment of IDH1.R132H MAF in RNA compared to DNA across all glioma subtypes (FIG. 1b), with the highest RNA/DNA enrichment in oligodendrogliomas followed by high grade and low grade astrocytomas (Average fold change, 8.5, 5.6 and 2.8 respectively) (FIG. 1b). Finally, we investigated the spatial variation of RNA/DNA enrichment ratio within different regions of tumor lesion (FIG. 1c). A total of n=3 patients (oligodendroglioma=2, astrocytoma=1) and 12 regions (4 per patient) were analyzed. Again, on average, spatial analysis of oligodendrogliomas demonstrated 22-fold RNA/DNA enrichment (95% CI: 13.49%-30.97%) as compared to 2.5-fold change in astrocytomas (95% CI: 1.36%-3.62%) (FIG. 1c).

Extending this comparison to biofluids, we tested 5 plasma samples consisting of IDH1.R132H mutant patients (n 3) and healthy controls (n=2). Parallel processing of 2 ml was performed to isolate either i) EV RNA (EXOLUTION, see Methods), or ii) EV RNA+EV DNA+cfDNA (EXOLUTIONPLUS, see Methods). There was a striking difference in the detection of IDH1.R132H (FIG. 1d, top panel) and IDH1 wild-type signals (FIG. 1d, bottom panel), with detection of mutant signals in all three patients using EV RNA analyte. However, no IDH1.R132H signal was detected using the DNA based approach (FIG. 1d, top panel) in addition to yielding a significantly lower IDH1 wild-type signal (IDH1wt) in patients and controls (FIG. 1d, bottom panel).

We next optimized downstream reverse transcription (FIG. 8e) and droplet digital PCR (ddPCR) conditions (annealing temperature, preamplification supermix; FIGS. 8a-e). In order to improve sensitivity in low input plasma samples, three different preamplification conditions were tested using 1.0 ng RNA input: i) Forward and Reverse primers only, ii) Primers+Mutant probe+Wild-type probe, iii) Primers and Mutant probe (FIG. 1e). Highest IDH1.R132H MAF was seen with condition (iii) with the difference being statistically significant. Next, the optimal preamplification condition was tested using IDH1 wild-type tumor tissue (RNA, 2 ng) and healthy control plasma (2 ml) with and without the presence of a previously designed wild-type blocker (see Methods, FIG. 8f) at an optimized concentration (FIG. 8f). The presence of wild-type blocker during preamplification facilitated the elimination of false positive signals in both control samples, thereby improving signal-to-noise ratio (FIG. 1f). Similar testing also demonstrated a favorable effect in reducing the IDH1wt signal in IDH1.R132H mutant plasma samples (FIG. 8f).

The optimized ddPCR assay (hereinafter termed mt-IDH1_{dx} assay) was tested to determine linearity and limit of detection (LOD)/Limit of Blank (LOB). A range of different concentrations of IDH1.R132H RNA (10.0-0.02) ng were spiked into a constant IDH1 wild-type background RNA (10.0 ng) prior to reverse transcription (FIG. 1g-h). The final ddPCR was performed in three replicates at each concentration. A linear trend was observed in the IDH1.R132H signal with an R² of 0.99, LOD of 0.06%, and LOB of 0.02% (FIG. 1g-h).

### Example 2. mt-IDH1_{dx} Assay Testing in IDH1.R132H Mutant Tumor Tissue Demonstrates Diagnostic and Prognostic Utility

As current glioma tumor biopsy to determine IDH1mt status is invasive, time intensive, costly, and frequently lacks sensitivity, we then tested the proposed mt-IDH1_{dx} assay (FIG. 1) in a variety of clinical samples to assess assay performance in both tissue and liquid samples and compared these results to clinically reported results. (FIG. 2a).

The mt-IDH1_{dx} assay performance was first evaluated in a cohort of tumor tissue (n=48; FIG. 2b) consisting of astrocytomas, grade 2-3 (n=25), oligodendrogliomas, grade 2-3 (n=11), and astrocytomas, grade 4 (n=12). The IDH1mt status was determined by MGH clinical pathology in astrocytomas, grade 2-3 (n=21), oligodendrogliomas, grade 2-3 (n=11), and astrocytomas, grade 4 (n=7) (FIG. 2b) and confirmed by the mt-IDH1_{dx} assay (100% concordance) (FIG. 2c-e).

Interestingly, the mt-IDH1_{dx} assay also detected the IDH1mt signal in an additional subset of Grade 2-3, Astrocytomas (n=2, FIG. 2c) and Grade 4, Astrocytomas (n=1, FIG. 2d) lesions that were classified as IDH1.R132H wt by MGH clinical pathology. The low grade astrocytomas were classified as IDH1.R132C and IDH1.R132S mutant by histopathology testing.

Distribution analysis of the IDH1mt copies (FIG. 2f), IDH1wt copies (FIG. 2g), and IDH1.R132H MAF (FIG. 2h) in tumor tissue suggested a significantly higher IDH1mt expression level in oligodendrogliomas (mean 1,926±1,373 copies, 95% CI. 1,004-2,848) followed by astrocytomas, grade 4 (mean: 963±1,716 copies, 95% CI: 963-2,053) and astrocytomas, grade 2-3 (mean 698±672 copies, 95% CI: 420-975) (FIG. 2f). IDH1wt was found to be highly overexpressed in grade 4 astrocytoma (Glioblastoma, based on previous WHO classification) tumor tissue (mean 3,404±3,351 copies, 95% CI: 1,275-5,534), as compared to low grade astrocytomas (mean 1,436±844) and oligodendrogliomas (mean 2,125±1,200) (FIG. 2g). Finally, IDH1.R132H MAF reflecting mutational expression, was highest in oligodendrogliomas tumors (mean: 44.5%±12.6, 95% CI: 36.0%-52.9%), followed by low grade astrocytomas (mean: 27.2%±16.3, 95% CI: 20.1%-33.9%) and high grade astrocytomas (mean: 20.4%±20, 95% CI: 7.7%-33.1%) (FIG. 2h).

Following signal quantification in the different clonal subtypes, Kaplan-Meier analysis was performed to compare the overall prognosis in patients with different patterns of IDH1mt detection. Median Overall Survival (OS) was determined to be highest in the oligodendroglioma subtype (132.8 months) followed by grade 2-3, astrocytoma (101.5 months) and grade 4, astrocytoma subtype (52.6 months; FIG. 2i). Correlation of the OS to MAF distribution (FIG. 2h), demonstrated a positive correlation of tissue IDH1.R132H MAF with overall survival, with highest tumor tissue IDH1.R132H MAF in oligodendrogliomas also associated with highest overall survival. Similarly, the subtype with lowest MAF (astrocytoma, grade 4) indicated significantly shorter overall survival. To investigate this relationship further, we stratified the patient population into two groups based on ddPCR derived IDH1.R132H tissue MAF cut-off (≤38%, >38%; FIG. 2j) determined by the overall distribution in the population. Importantly, this analysis also demonstrated a survival benefit in the group with higher IDH1.R132H MAF (>38%) with approximately 2-fold higher median OS (130.1 months) compared to the group with lower MAF (≤38%: 75.8 months) (FIG. 2j). Additional survival analysis based on disease parameters was also performed.

The experiments were performed using both the Bio-Rad and QIAcuity systems with similar results.

### Example 3. Clinical Validation of the Mt-IDH1_{dx} Assay Performance in EV RNA Derived from Patient Plasma

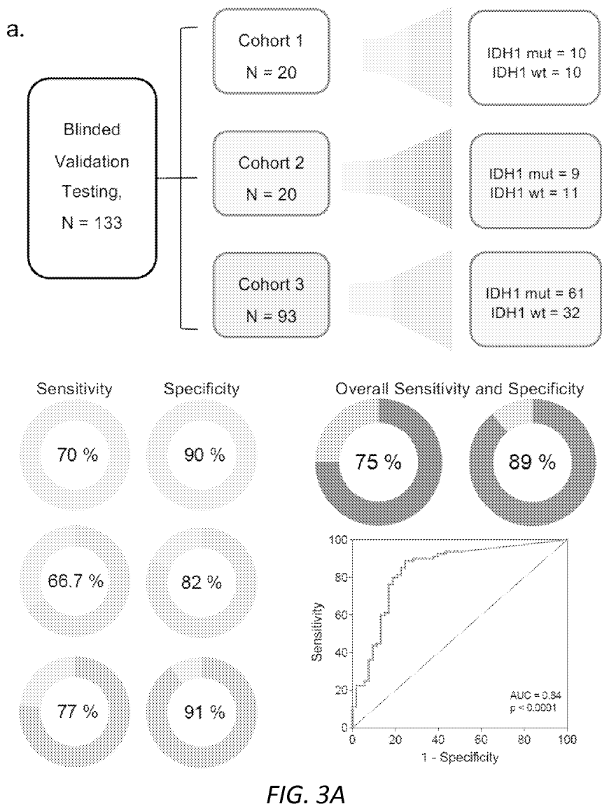

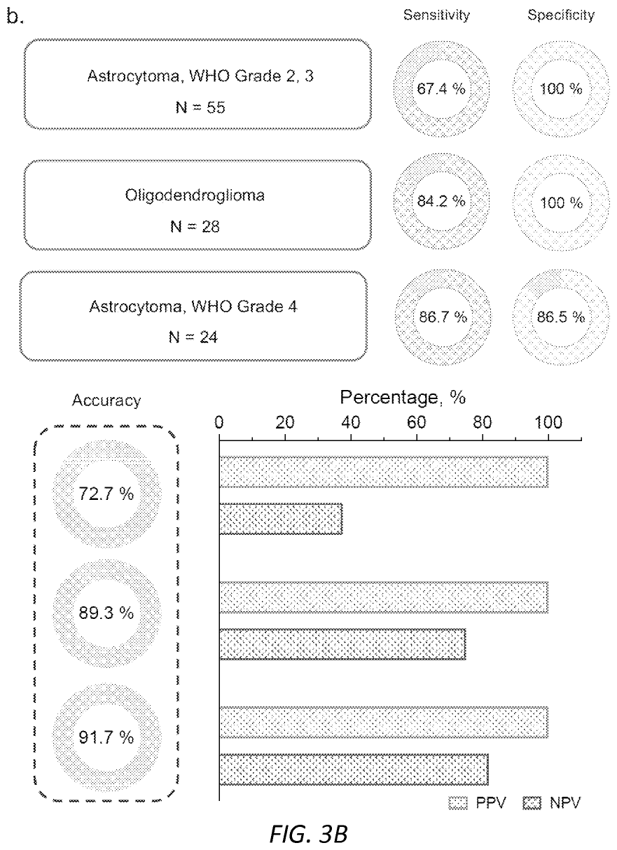

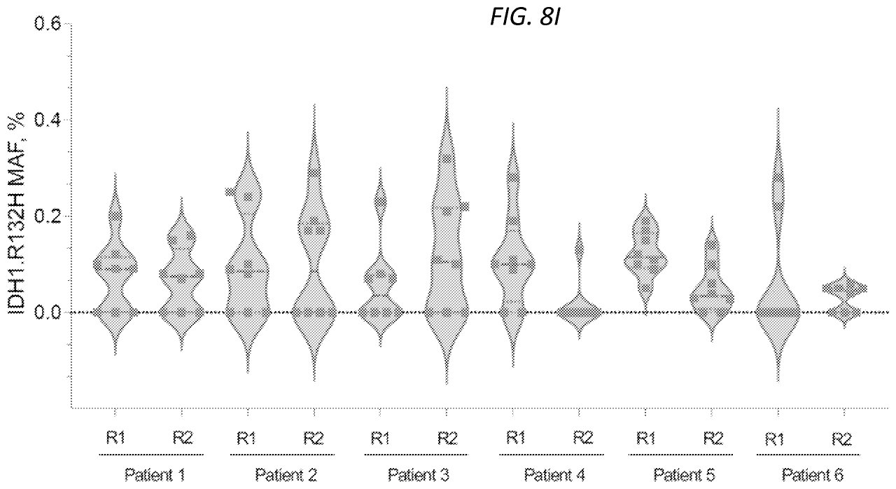

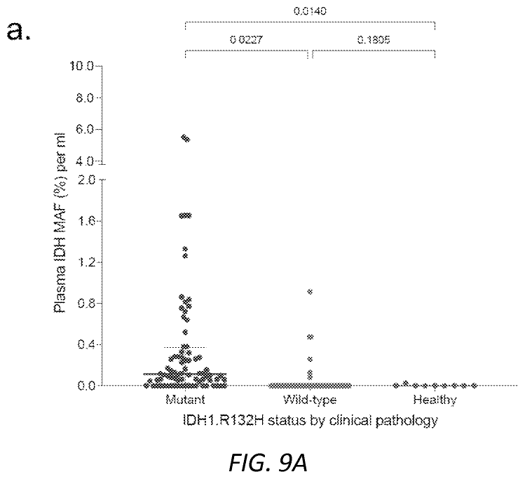

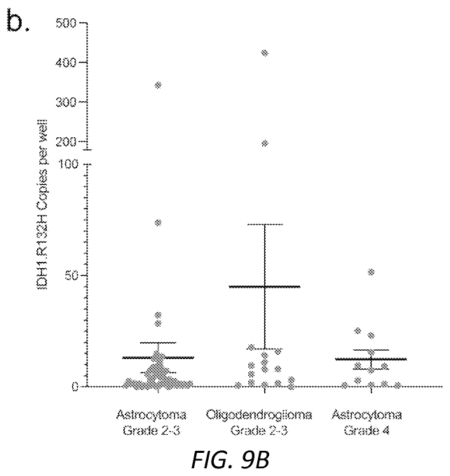

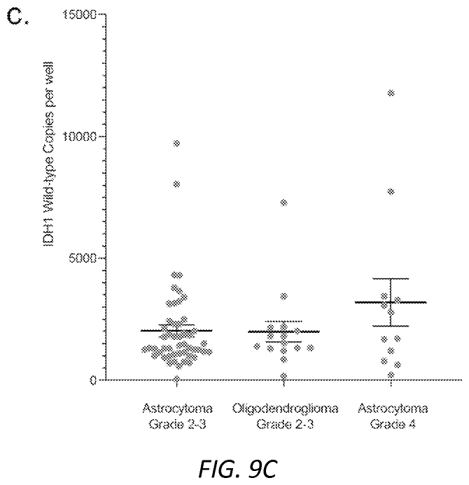

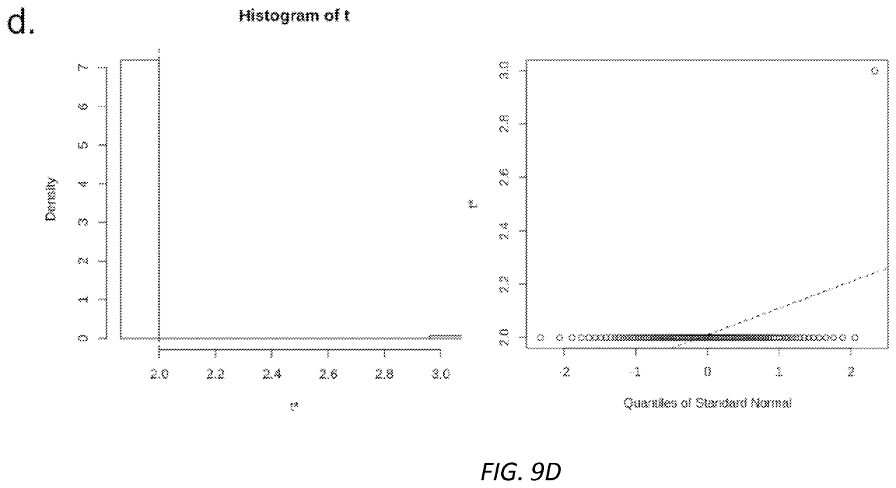

mt-IDH1_{dx} assay performance was evaluated in plasma samples across three blinded, validation cohorts (N=133, Cohort 1; n=20, Cohort 2; n=20, Cohort 3; n=93) (FIG. 3a) comprising histopathology confirmed IDH1mt glioma patients (n=80), IDH1wt glioma patients (n=44) and age matched healthy controls (n=9) (FIG. 3a). Two ml of plasma was processed from each sample (see Methods) and eight replicates analyzed at the final ddPCR step. The cut-off (i.e., number of positive replicates) for calling a sample positive was statistically determined by calculation of the Youden index (FIG. 9d). Based on this calculation and clinical relevance, samples having at least three (≥3) positive replicates with IDH1.R132H MAF % greater than or equal to 0.06% based on Limit of Detection (LOD) experiment, were considered IDH1mt positive.

Baseline plasma testing results were as follows (FIG. 3a): Cohort 1; Sensitivity 70.0% (95% CI: 34.8%-93.3%), Specificity 90.0% (95% CI: 55.5%-99.8%), Cohort 2; Sensitivity 66.7% (95% CI: 29.9%-92.5%), Specificity 81.8% (95% CI: 48.2%-97.7%), Cohort 3; Sensitivity 77.1% (95% CI: 64.5%-86.9%), Specificity 90.6% (95% CI: 75.0%-98.0%) (FIG. 3a). Overall, across the study population (N=133), our mt-IDH1_{dx} assay detected IDH1.R132H mutation in plasma derived EV RNA with an overall sensitivity of 75.0% (95% CI: 64.1%-84.0%), Specificity 88.7% (95% CI: 77.0%-95.7%), and AUC 0.84 (p<0.0001) (FIG. 3a). Positive predictive value (PPV) and negative predictive value (NPV) was determined to be 90.9% (95% CI: 82.3%-95.6%) and 70.1% respectively (61.4%-77.7%) (FIG. 3a). In a subset (n=9) IDH1wt (by MGH clinical pathology) patient samples, IDH1mt signal was detected by plasma testing: Grade 4, Astrocytoma (Secondary GBM; n=3), Primary GBM (n=4), Multifocal GBM (n=1) and low grade Glioneuronal tumor (n=1). Subsequent mt-IDH1_{dx} assay testing of tumor tissue (available in n=3) derived RNA confirmed the presence of the IDH1mt signal. Of note, in the cases where plasma mt-IDH1_{dx} testing was positive but clinical pathology reporting was negative, the pathology assessment of IDH1mt in the above cases (including n=3 validated by mt-IDH1_{dx} ddPCR testing) was clinically determined by Immunohistochemistry (IHC). Clinical information and demographics variables are summarized for IDH1mt (Table 1A) and IDH1wt (Table 1B) study population included in each individual cohort. Additional correlation analysis of EV RNA IDH1.R132H MAF % with various clinical and disease parameters was performed across different glioma subtypes.

In order to evaluate the blood-based mt-IDH1_{dx} assay performance across different histological subtypes of IDH1mt gliomas, the study population was stratified into three groups (FIG. 3b): i) Astrocytoma, Grade 2-3 (N=55), ii) Oligodendroglioma, Grade 2-3 (N=28), and iii) Astrocytoma, Grade 4 (N=24). The assay was able to perform robust sensitive detection of common IDH1mt gliomas, i.e., low grade astrocytomas (67.4%, 95% CI: 52.0%-80.5%) and oligodendrogliomas (84.2%, 95% CI: 60.4%-96.6%) with a specificity of 100% (95% CI: 63.7%-100%) (FIG. 3b). Furthermore, the mt-IDH1_{dx} assay can also be used in conjunction with the radiological imaging as a companion diagnostic to accurately differentiate between the common IDH1mt glioma subtypes (Astrocytomas, grade 2-3: 72.7%, Oligodendroglioma, grade 2-3: 89.3%, Astrocytoma, grade 4: 73.0%).

### Example 4. Prognostication of the Mt-IDH1_{dx} Assay Across Glioma Subtypes

In addition to baseline clinical diagnostics, we also explored the viability of using blood-based detection of IDH1.R132H as a prognostication tool to predict survival outcomes. Cohorts described in FIG. 3 (Cohort 1, 2, 3) were combined here to perform prognostication analysis. Patient population stratification via plasma based EV RNA IDH1.R132H MAF, % (<0.15, ≥0.15) demonstrated an overall survival benefit in patients with higher (≥0.15%, 125.4 vs 87.2 months) mutational expression (FIG. 4a). The results indicating improved survival outcomes in lesions with higher MAF were in line with the previous observations made in the Kaplan-Meier analysis of tumor tissue cohort (FIG. 2j).

Next, EV RNA IDH1.R132H MAF % distribution was compared across the three glioma subtypes (FIG. 4b). Interestingly, a similar pattern of IDH1mt enrichment was noted in the three histological variants in both tumor tissue (FIG. 2h) and plasma derived EV mRNA (FIG. 4b). Oligodendroglioma subtype was associated with a 2-fold higher enrichment of IDH1mt EV RNA than astrocytoma, grade 2-3 (Oligodendroglioma: 1.28±0.71, Astrocytoma, grade 2-3: 0.63±0.32) and 3-fold higher enrichment than astrocytoma, grade 4 (0.38±0.14) (FIG. 4b). An independent comparison of overall survival (OS) in the same groups demonstrated a significantly higher median OS (87.2 months-102.6 months) in subtypes expressing higher IDH1 MAF (low grade astrocytoma, oligodendroglioma) versus the subtype with lower MAF (high grade astrocytoma, 50.4 months) (FIG. 4c), again demonstrating a positive prognostic correlation between IDH1 MAF and overall survival (OS).

Finally, we determined the prognostication utility of EV RNA IDH1.R132H MAF, % in the glioma subtypes at cut-offs determined by population distribution in each group (FIG. 4d-f). In the low-grade astrocytoma group, no difference in short-term progression free survival (PFS) was found between the two curves. However, there was a significant difference in long-term PFS (9.3 yr, 11 yr) with a longer median PFS seen in patients with higher (≥0.08%) EV RNA IDH1.R132H MAF determined at baseline diagnostics (FIG. 4d). Patient stratification via EV RNA IDH1.R132H MAF (<0.26%, ≥0.26%) in oligodendroglioma group demonstrated a much larger difference (208 vs. 74 months) in PFS with higher MAF (≥0.26%) conferring a better prognosis (FIG. 4e). The difference between the two survival curves was shown to be statistically significant at 6.2 yr and 7.3 yr follow-up timepoints (FIG. 4e). In the more aggressive subtype, i.e., grade 4 astrocytoma, a similar pattern was observed with higher MAF (≥0.12%) being a favorable prognostic factor (FIG. 4f). A higher baseline EV RNA IDH1.R132H MAF was associated with a 3-fold longer median PFS (91.4 vs. 50.4 months). Importantly, this glioma subtype has previously been reported to have poor survival outcomes versus low grade astrocytoma and oligodendroglioma. Interestingly, in our plasma-based prognostication analysis, the EV RNA IDH1.R132H MAF cut-off was an important tool in predicting short term PFS at 3.9 yr and 5.2 yr mark (versus longer follow-up timepoints in other subtypes), indicating the potential significance in a more rapidly progressing tumor lesion (FIG. 4f).

Overall, a higher plasma EV RNA IDH1.R132H MAF, % was identified as a favorable prognostic factor for predicting improved survival outcomes across all clonal subtypes. This is consistent with earlier findings of positive correlation between tumor tissue MAF and overall survival in an independent cohort.

### Example 5. Higher Tumor Analyte Shedding Rate in Plasma Correlates with Improved Overall Survival

A subset of patients (tested in FIG. 3) was selected where matched tissue and plasma analysis was performed. The mt-IDH1_{dx} assay was performed in matched tumor tissue and plasma samples (n=24) across three IDH1mt glioma subtypes: Astrocytoma, grade 2-3 (n=14), Oligodendroglioma, grade 2-3 (n=5), and Astrocytoma, grade 4 (n=5). Statistical evaluation of the differential expression of IDH1mt (FIG. 5a) and IDH1wt (FIG. 5b) in tumor tissue versus. plasma was performed via plotting log fold-change (FC, Log₂ ratio=Avg. Mut/WT, Plasma/Avg. Mut/WT, Tissue) (y-axis) against average expression (x-axis; Average, Mut/Wt tissue and Mut/Wt plasma) of mutant (FIG. 5a) and wild-type (FIG. 5b) targets. We observed considerable variation in tissue versus plasma enrichment of IDH1mt RNA across the three clonal subtypes (FIG. 5a). On the contrary, IDH1 wild-type RNA showed similar expression levels in tumor tissue derived RNA and plasma derived EV RNA across the low grade and high grade gliomas (FIG. 5b).

Among patient samples with higher enrichment of the IDH1mt signal in plasma, Grade 3 Astrocytomas (n=4, P2, P8-9, P11) were shown to demonstrate the highest shedding rate than other subtypes (FIG. 5a, encircled in red). Comparing all subtypes, low grade astrocytomas were associated with a 3-fold higher enrichment (Log₂ FC: 6.5±3.1) of IDH1mt signal in EV RNA versus the more aggressive grade 4 subtype (Log₂ FC: 2.0±1.8) (FIG. 5a). Importantly, tumor lesions demonstrating higher shedding rate as statistically determined by M value (enrichment in plasma) were also associated with an improved overall survival (OS). In this case, the difference in median OS between low vs. high grade astrocytomas was statistically significant (p=0.038, *) with a 2-fold longer OS in higher shedding grade 2-3, astrocytoma (129.5 months) than the lower shedding grade 4, astrocytoma lesions (65.6 months).

Tumor tissue enrichment was noted for (n=9) astrocytomas with an average M value of 1.98 (0.25-4.59) (FIG. 5a). To compare the clinical outcomes and prognosis of astrocytomas with higher versus lower shedding rates, Kaplan Meier analysis was performed. A statistically significant difference (p=0.03, *) was found between the two survival curves. Astrocytomas demonstrating higher shedding of EV mRNA (n=5) were associated with a longer median OS (129.5 months) than the lesions less likely to shed (n=9, 72.2 months).

Next, based on previous observations of IDH1.R132H MAF distribution among different patient characteristics (independent tissue and plasma analysis), we proceeded to test this in the matched tumor tissue (FIG. 5c, top) and plasma samples (FIG. 5c, bottom). Overall, similar trends were seen in both analyses (FIG. 5c), with higher IDH1.R132H MAF (tissue and EV RNA) reported in: i) age group>40 yr, ii) tumors in frontal lobe, and iii) lesions in dominant (left) hemisphere. Finally, consistent with previous findings, IDH1.R132H MAF served as a positive prognostic factor in tumor tissue and plasma, with higher baseline MAF (tissue: >38%, plasma: >0.38%) associated with a longer median OS (FIG. 5c).

### Example 6. Longitudinal Glioma Monitoring Using the Mt-IDH1_{dx} Assay, Correlates with Radiological and Clinical Outcomes

In the final phase of clinical validation, we tested the utility of mt-IDH1_{dx} in longitudinal disease monitoring and surveillance. A cohort of n=8 plasma samples (subset of the patients tested in FIG. 3) was included with evaluation at multiple clinical timepoints: tumor progression, pre-, early-, and post-therapy, MRI surveillance, and stable disease (FIG. 6). The study population (n=8) was divided into three groups based on different disease outcomes: i) Disease Progression (P1, P2, P7, FIG. 6a), ii) Treatment Response (P3, P4, P6, FIG. 6b), and iii) Stable (or Minimal Residual) Disease (P5, P8, FIG. 6c).

In the disease progression group (P1, P2, P7, FIG. 6a), different glioma subtypes were represented: P1 (Astrocytoma, Grade 2), P2 (Astrocytoma, Grade 3), P7 (Astrocytoma, Grade 4). Across all time points, P1 showed rising plasma levels of IDH1.R132H MAF, consistent with disease progression as evidenced by surveillance MRI and neurological deterioration. Radiological imaging performed shortly before the second plasma sampling, was inconclusive for pseudo-versus true tumor progression. However, subsequent tumor tissue testing (stereotactic biopsy) confirmed progression to grade 4 lesion, and this was consistent with the blood-based findings. Similarly, in both P2 and P7, the plasma based EV RNA IDH1.R132H MAF remained high and was consistent with progressive disease (according to MRI findings), correlating with worsening clinical outcomes.

In the treatment response group (P3, P4, P6, FIG. 6b), across all three patients, we observed a steep decline in blood-based IDH1.R132H MAF, with plasma levels dropping to undetectable levels in ⅔ at the last follow-up time-point available to us. This was in perfect concordance with the radiological imaging findings indicating stable disease and good treatment response. Interestingly, in all three patients we observed an early spike in the IDH1mt plasma-levels following treatment initiation (P3: TMZ, P4: PCV, P6: AG-881) followed by progressively declining IDH1mt signal over the course of treatment. In P6, the short-term follow-up plasma testing demonstrated a very low, but persistent IDH1mt signal. Importantly, this correlated with the MRI findings of unchanged T2/FLAIR abnormality which remains under surveillance and has not progressed (based on last clinical follow-up).

In the stable disease group (P5, P8, FIG. 6c), we observed similar concordance of plasma testing and clinical evaluation with P5 and P8 presenting with an overall stable disease. Surveillance MRI performed during the treatment course of P5 was inconclusive at first for pseudo progression versus treatment related effects. However, pseudo progression was later confirmed based on a decrease in contrast enhancement and T2/FLAIR hyperintensity. These findings were concordant with the drop in IDH1mt signal measured in plasma testing. P8 also had an initial increase in IDH1.R132H MAF corresponding to tumor progression on MRI. Follow-up plasma testing correlated with treatment response and stable disease on surveillance imaging.

Interestingly, a consistent increase in the plasma IDH1mt levels was observed in response to various treatment modalities. This is summarized (FIG. 6d) for multiple patients by performing normalization of EV RNA IDH1.R132H MAF (see Methods) and comparing the signal at three time-points: pre-, early-, and post-therapy. This potentially highlights an important biological process of tumor shedding and release of tumor derived EVs into systemic circulation, following treatment initiation.

### REFERENCES

1. Lin, D. et al. Trends in Intracranial Glioma Incidence and Mortality in the United States, 1975-2018. Front. Oncol. 11, 748061 (2021).
2. Yan, H. et al. IDH1 and IDH2 mutations in gliomas. N. Engl. J. Med. 360, 765-773 (2009).
3. Dang, L. et al. Cancer-associated IDH1 mutations produce 2-hydroxyglutarate. Nature 462, 739-744 (2009).
4. Nobusawa, S., Watanabe, T., Kleihues, P. & Ohgaki, H. IDH1 mutations as molecular signature and predictive factor of secondary glioblastomas. Clin. Cancer Res. 15, 6002-6007 (2009).
5. Hertler, C. et al. Long-term survival with IDH wildtype glioblastoma: first results from the ETERNITY Brain Tumor Funders' Collaborative Consortium (EORTC 1419). Eur. J. Cancer 189, 112913 (2023).
6. Hooper, G. W., Ansari, S., Johnson, J. M. & Ginat, D. T. Advances in the Radiological Evaluation of and Theranostics for Glioblastoma. Cancers 15, (2023).
7. Katzendobler, S. et al. Diagnostic Yield and Complication Rate of Stereotactic Biopsies in Precision Medicine of Gliomas. Front. Neurol. 13, 822362 (2022).
8. van den Bent, M. J. Interobserver variation of the histopathological diagnosis in clinical trials on glioma: a clinician's perspective. Acta Neuropathol. 120, 297-304 (2010).
9. Zhu, P. et al. The detrimental effect of biopsy preceding resection in surgically accessible glioblastoma: results from the national cancer database. J. Neurooncol. (2024) doi:10.1007/si 1060-024-04644-z.
10. Eibl, R. H. & Schneemann, M. Liquid Biopsy and Primary Brain Tumors. Cancers 13, (2021).
11. Batool, S. M. et al. The Liquid Biopsy Consortium: Challenges and opportunities for early cancer detection and monitoring. Cell Rep Med 4, 101198 (2023).
12. Batool, S. M. et al. Extrinsic and intrinsic preanalytical variables affecting liquid biopsy in cancer. Cell Rep Med 4, 101196 (2023).
13. Muralidharan, K. et al. Promoter Mutation Analysis for Blood-Based Diagnosis and Monitoring of Gliomas. Clin. Cancer Res. 27, 169-178 (2021).
14. Batool, S. M. et al. Highly Sensitive EGFRvIII Detection in Circulating Extracellular Vesicle RNA of Glioma Patients. Clin. Cancer Res. 28, 4070-4082 (2022).
15. Figueroa, J. M. et al. Detection of wild-type EGFR amplification and EGFRvIII mutation in CSF-derived extracellular vesicles of glioblastoma patients. Neuro. Oncol. 19, 1494-1502 (2017).
16. Chen, W. W. et al. BEAMing and Droplet Digital PCR Analysis of Mutant IDH1 mRNA in Glioma Patient Serum and Cerebrospinal Fluid Extracellular Vesicles. Mol. Ther. Nucleic Acids 2, e109 (2013).
17. Louis, D. N. et al. The 2007 WHO classification of tumours of the central nervous system. Acta Neuropathol. 114, 97-109 (2007).
18. Louis, D. N. et al. The 2016 World Health Organization Classification of Tumors of the Central Nervous System: a summary. Acta Neuropathol. 131, 803-820 (2016).
19. Wen, P. Y. & Packer, R. J. The 2021 WHO Classification of Tumors of the Central Nervous System: clinical implications. Neuro-oncology vol. 23 1215-1217 (2021).
20. Shen, G. et al. The MRI Features and Prognosis of Gliomas Associated With IDH1 Mutation: A Single Center Study in Southwest China. Front. Oncol. 10, 852 (2020).
21. Joyner, D. A. et al. MRI features predict tumor grade in isocitrate dehydrogenase (IDH)-mutant astrocytoma and oligodendroglioma. Neuroradiology 65, 121-129 (2023).
22. Cabezas-Camarero, S. et al. Detection of IDH1 Mutations in Plasma Using BEAMing Technology in Patients with Gliomas. Cancers 14, (2022).
23. Boisselier, B. et al. Detection of IDH1 mutation in the plasma of patients with glioma. Neurology 79, 1693-1698 (2012).
24. Husain, A., Mishra, S., Siddiqui, M. H. & Husain, N. Detection of IDH1 Mutation in cfDNA and Tissue of Adult Diffuse Glioma with Allele-Specific qPCR. Asian Pac. J. Cancer Prev. 24, 961-968 (2023).
25. Fujita, Y. et al. IDH1 p.R132H ctDNA and D-2-hydroxyglutarate as CSF biomarkers in patients with IDH-mutant gliomas. J. Neurooncol. 159, 261-270 (2022).
26. Tuna, G. et al. Minimally Invasive Detection of IDH1 Mutation with Cell-Free Circulating Tumor DNA and D-2-Hydroxyglutarate, D/L-2-Hydroxyglutarate Ratio in Gliomas. J. Neuropathol. Exp. Neurol. 81, 502-510 (2022).
27. Landers, M. J. F. et al. Oligodendrogliomas tend to infiltrate the frontal aslant tract, whereas astrocytomas tend to displace it. Neuroradiology 65, 1127-1131 (2023).
28. Cancer Genome Atlas Research Network et al. Comprehensive, Integrative Genomic Analysis of Diffuse Lower-Grade Gliomas. N. Engl. J. Med. 372, 2481-2498 (2015).
29. DeWitt, J. C. et al. Cost-effectiveness of IDH testing in diffuse gliomas according to the 2016 WHO classification of tumors of the central nervous system recommendations. Neuro. Oncol. 19, 1640-1650 (2017).
30. Bhaduri, A. et al. Outer Radial Glia-like Cancer Stem Cells Contribute to Heterogeneity of Glioblastoma. Cell Stem Cell 26, 48-63.e6 (2020).
31. Neftel, C. et al. An Integrative Model of Cellular States, Plasticity, and Genetics for Glioblastoma. Cell 178, 835-849.e21 (2019).
32. Tirosh, I. et al. Single-cell RNA-seq supports a developmental hierarchy in human oligodendroglioma. Nature 539, 309-313 (2016).
33. Venteicher, A. S. et al. Decoupling genetics, lineages, and microenvironment in IDH-mutant gliomas by single-cell RNA-seq. Science 355, (2017).
34. Nicholson, J. G. & Fine, H. A. Diffuse Glioma Heterogeneity and Its Therapeutic Implications. Cancer Discov. 11, 575-590 (2021).
35. Vij, M. et al. The prognostic impact of subclonal IDH1 mutation in grade 2-4 astrocytomas. Neurooncol Adv 5, vdad069 (2023).
36. Carstam, L. et al. Long-term follow up of patients with WHO grade 2 oligodendroglioma. J. Neurooncol. 164, 65-74 (2023).
37. Nn5ez, K. G. et al. Exosome shedding is concordant with objective treatment response rate and stratifies time to progression in treatment naïve, non-resectable hepatocellular carcinoma. Livers 3, 727-738 (2023).
38. Andrade, L. N. de S. et al. Extracellular Vesicles Shedding Promotes Melanoma Growth in Response to Chemotherapy. Sci. Rep. 9, 14482 (2019).
39. Gastpar, R. et al. Heat shock protein 70 surface-positive tumor exosomes stimulate migratory and cytolytic activity of natural killer cells. Cancer Res. 65, 5238-5247 (2005).
40. Scholl, J. N. et al. Characterization and antiproliferative activity of glioma-derived extracellular vesicles. Nanomedicine 15, 1001-1018 (2020).
41. Gromeier, M. et al. Very low mutation burden is a feature of inflamed recurrent glioblastomas responsive to cancer immunotherapy. Nat. Commun. 12, 352 (2021).
42. Malta, T. M. et al. The Epigenetic Evolution of Glioma Is Determined by the IDH1 Mutation Status and Treatment Regimen. Cancer Res. 84, 741-756 (2024).
43. Choi, B. D. et al. Intraventricular CARv3-TEAM-E T Cells in Recurrent Glioblastoma. N. Engl. J. Med. 390, 1290-1298 (2024).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for detecting a mutation at position R132 in Isocitrate Dehydrogenase 1 gene (IDH1) in a sample comprising a biofluid from a subject, the method comprising:
providing a sample comprising ribonucleic acids isolated from extracellular vesicles (EV RNA) extracted from the sample, reverse transcribing the EV RNA to generate EV cDNA;
performing a preamplification reaction by contacting the EV cDNA with a pair of amplification primers comprising a forward primer and a reverse primer, in the presence of a wild-type blocking oligonucleotide;
performing a further amplification reaction by contacting the preamplified sample with the pair of amplification primers comprising a forward primer and a reverse primer, and a pair of detection primers comprising a mutant primer and a wild-type primer, wherein the mutant primer comprises a first detectable label and the wild-type primer comprises a second detectable label, under conditions sufficient for amplifying the IDH1 sequence, and
detecting a signal from the first detectable label and/or the second detectable label, wherein presence of the signal from the first detectable label indicates presence of a mutant IDH1 sequence in the sample and/or wherein presence of the signal from the second detectable label indicates presence of a wild-type IDH1 promoter sequence in the sample.

2. The method of claim 1, wherein the mutation is R132H, and:
the forward primer comprises SEQ ID NO: 1 and the reverse primer comprises SEQ ID NO:2;
the mutant primer comprises SEQ ID NO: 4 or 24 and the wild-type primer comprises SEQ ID NO: 5 or 25; and/or
the wild-type blocking oligonucleotide comprises SEQ ID NO:8.

3. The method of claim 1, wherein the further amplification reaction is droplet digital PCR (ddPCR).

4. The method of claim 1, wherein the sample comprises serum, saliva, urine, or cerebrospinal fluid.

5. A method for detecting a mutation at position R132 in Isocitrate Dehydrogenase 1 gene (IDH1) in a sample comprising tumor cells or tissue from a subject, the method comprising:
providing a sample comprising ribonucleic acids isolated from the sample, reverse transcribing the RNA to generate cDNA;
performing a preamplification reaction by contacting the cDNA with a pair of amplification primers comprising a forward primer and a reverse primer, in the presence of a wild-type blocking oligonucleotide;
performing a further amplification reaction by contacting the preamplified sample with the pair of amplification primers comprising a forward primer and a reverse primer, and a pair of detection primers comprising a mutant detection primer and a wild-type detection primer, wherein the mutant primer comprises a first detectable label and the wild-type primer comprises a second detectable label, under conditions sufficient for amplifying the IDH1 sequence, and
detecting a signal from the first detectable label and/or the second detectable label, wherein presence of the signal from the first detectable label indicates presence of a mutant IDH1 sequence in the sample and/or wherein presence of the signal from the second detectable label indicates presence of a wild-type IDH1 promoter sequence in the sample.

6. The method of claim 5, wherein the mutation is R132H, and:
the forward primer comprises SEQ ID NO: 1 and the reverse primer comprises SEQ ID NO:2;
the mutant primer comprises SEQ ID NO: 4 or 24 and the wild-type primer comprises SEQ ID NO: 5 or 25; and/or
the wild-type blocking oligonucleotide comprises SEQ ID NO:8.

7. The method of claim 5, wherein the further amplification reaction is droplet digital PCR (ddPCR).

8. The method of claim 1, wherein the wild type blocking oligonucleotide comprises at least one locked nucleic acid (LNA) modification.

9. The method of claim 1, wherein the detection primers each comprise a fluorophore and a quencher.

10. The method of claim 9, wherein the mutant detection primers comprise a first fluorophore and a first quencher, and the second detection primers comprise a second fluorophore and a second quencher.

11. The method of claim 10, wherein the first fluorophore and the second fluorophore are different fluorophores, and/or wherein the first quencher and the second quencher are the same quencher.

12. The method of claim 10, wherein the first fluorophore and the second fluorophore are selected from the group consisting of FAM, HEX, Cy3, Cy5, and Texas Red, and/or wherein the first quencher and the second quencher are selected from the group consisting of Iowa Black FQ, Iowa Black RQ, ZEN Quencher, and TAMRA.

13. The method of claim 1, wherein the subject is a human patient having or suspected of having a cancer.

14. The method of claim 13, wherein the cancer is selected from glioma, myeloid malignancies, chondrosarcoma, cholangiocarcinoma, astrocytoma, secondary glioblastoma (diffuse astrocytoma), ependymoma, primary glioblastoma, cartilaginous and bone tumors, intrahepatic cholangiocarcinoma, acute myeloid leukemia.

15. The method of claim 14, wherein the brain cancer is a glioma, optionally wherein the glioma is an astrocytoma, an ependymoma, or an oligodendroglioma, or the myeloid malignancy is acute myeloid leukemia (AML) and myelodysplastic syndromes (MDS).

16. The method of claim 1, further comprising administering a cancer therapy to the subject when the subject has been identified as having an IDH1 mutation.

17. The method of claim 16, wherein the cancer therapy is selected from the group consisting of a chemotherapy (optionally an IDH-targeted therapy), a radiation therapy, a surgical therapy, and an immunotherapy.

18. A method of evaluating reoccurrence of a cancer in a subject, the method comprising:
detecting the IDH1 sequence in a sample from the subject according to the method of claim 1,
determining a level of IDH1 mutant sequences in the sample and comparing the level to a reference level;
determining that the subject has reoccurrence of the cancer, when the subject is identified as having a level of IDH1 mutant sequences in the sample above the reference level, and optionally administering a cancer therapy to the subject identified as having a reoccurrence of the cancer.

19. A method of evaluating effectiveness of a cancer therapy, the method comprising:
in a sample from the subject who has been administered the cancer therapy, detecting the IDH1 sequence according to the method of claim 1,
determining a level of IDH1 mutant sequences in the sample and comparing the level to a reference level, optionally wherein the reference level is a level determined in the subject at an earlier time point, optionally before the administration of the therapy;
determining that the cancer therapy has been effective when the level of mutant IDH1 sequences in the sample is below the reference level, and optionally administering the cancer therapy identified as effective to the subject and/or administering another cancer therapy to the subject.

20. A kit for amplifying a region of a human IDH1 gene, the kit comprising a forward primer comprising SEQ ID NO: 1; a reverse primer comprising SEQ ID NO: 2; a mutant primer comprising SEQ ID NO: 4 or 24; a wild-type primer comprising SEQ ID NO: 5 or 25; and/or a wild-type blocking oligonucleotide comprising SEQ ID NO:8, and reagents for performing the method of claim 1.

21. The kit of claim 20, wherein the reagents are for performing EV isolation from serum and/or droplet digital PCR (ddPCR).

22. The kit of claim 20, wherein the wild type blocking oligonucleotide comprises at least one locked nucleic acid (LNA) modification.

23. The kit of any claim 20, wherein the detection primers each comprise a fluorophore and a quencher, optionally wherein the mutant detection primers comprise a first fluorophore and a first quencher, and the second detection primers comprise a second fluorophore and a second quencher.

24. The kit of claim 23, wherein the first fluorophore and the second fluorophore are different fluorophores, and/or wherein the first quencher and the second quencher are the same quencher, optionally wherein the first fluorophore and the second fluorophore are selected from the group consisting of FAM, HEX, Cy3, Cy5, and Texas Red, and/or wherein the first quencher and the second quencher are selected from the group consisting of Iowa Black FQ, Iowa Black RQ, ZEN Quencher, and TAMRA.


SEQUENCE LISTING (26 sequences)

SEQ ID NO: 1 - DNA, 17 bp, synthetic construct
  source 1..17: mol_type = other DNA
agtggatgggtaaaacc

SEQ ID NO: 2 - DNA, 17 bp, synthetic construct
  source 1..17: mol_type = other DNA
gatcaatacagagcaac

SEQ ID NO: 3 - DNA, 17 bp, synthetic construct
  source 1..17: mol_type = other DNA
ctagttatgtctcgttg

SEQ ID NO: 4 - DNA, 25 bp, synthetic construct
  source 1..25: mol_type = other DNA
  modified_base 1: mod_base = OTHER; note = 5'FAM fluorophore
  modified_base 8: mod_base = OTHER; note = locked nucleic acid
  modified_base 12: mod_base = OTHER; note = locked nucleic acid
  modified_base 16: mod_base = OTHER; note = locked nucleic acid
  modified_base 20: mod_base = OTHER; note = locked nucleic acid
  modified_base 25: mod_base = OTHER; note = IOWA BLACK FQ quencher
ntcatagngtcnatcnatgncttan

SEQ ID NO: 5 - DNA, 25 bp, synthetic construct
  source 1..25: mol_type = other DNA
  modified_base 1: mod_base = OTHER; note = 5'HEX fluorophore
  modified_base 8: mod_base = OTHER; note = locked nucleic acid
  modified_base 12: mod_base = OTHER; note = locked nucleic acid
  modified_base 16: mod_base = OTHER; note = locked nucleic acid
  modified_base 16: mod_base = OTHER; note = locked nucleic acid
  modified_base 20: mod_base = OTHER; note = locked nucleic acid
  modified_base 25: mod_base = OTHER; note = IOWA BLACK FQ quencher
ntcatagngtcngtcnatgncttan

SEQ ID NO: 6 - DNA, 18 bp, synthetic construct
  source 1..18: mol_type = other DNA
  modified_base 18: mod_base = OTHER; note = 3' Phosphorylation
cataggtctcatgcttan

SEQ ID NO: 7 - DNA, 23 bp, synthetic construct
  source 1..23: mol_type = other DNA
  modified_base 23: mod_base = OTHER; note = 3'Phosphorylation
ccccataagcatgagacctatgn

SEQ ID NO: 8 - DNA, 24 bp, synthetic construct
  source 1..24: mol_type = other DNA
  modified_base 24: mod_base = OTHER; note = 3'Phosphorylation
ccccataagcatgacgacctatgn

SEQ ID NO: 9 - DNA, 75 bp, synthetic construct
  source 1..75: mol_type = other DNA
ttgtgagtggatgggtaaaacctatcatcataggtcatcatgcttatggggatcaagtaagtcatgttggcaata

SEQ ID NO: 10 - DNA, 21 bp, synthetic construct
  source 1..21: mol_type = other DNA
tacagagcaactgattttgtt

SEQ ID NO: 11 - DNA, 17 bp, synthetic construct
  source 1..17: mol_type = other DNA
tcatgcttatggggatc

SEQ ID NO: 12 - DNA, 15 bp, synthetic construct
  source 1..15: mol_type = other DNA
tatcatcataggtca

SEQ ID NO: 13 - DNA, 16 bp, synthetic construct
  source 1..16: mol_type = other DNA
tatcatcataggtcat

SEQ ID NO: 14 - DNA, 17 bp, synthetic construct
  source 1..17: mol_type = other DNA
tatcatcataggtcatc

SEQ ID NO: 15 - DNA, 19 bp, synthetic construct
  source 1..19: mol_type = other DNA
tcataggtcatcatgctta

SEQ ID NO: 16 - DNA, 22 bp, synthetic construct
  source 1..22: mol_type = other DNA
atctgcaaaaatatcccccggc

SEQ ID NO: 17 - DNA, 17 bp, synthetic construct
  source 1..17: mol_type = other DNA
gatccccataaggatga

SEQ ID NO: 18 - DNA, 17 bp, synthetic construct
  source 1..17: mol_type = other DNA
agttgctctgtattgat

SEQ ID NO: 19 - DNA, 19 bp, synthetic construct
  source 1..19: mol_type = other DNA
agttgctctgtattgatcc

SEQ ID NO: 20 - DNA, 23 bp, synthetic construct
  source 1..23: mol_type = other DNA
acaacaaaatcagttgctctgta

SEQ ID NO: 21 - DNA, 15 bp, synthetic construct
  source 1..15: mol_type = other DNA
atcatcataggtcat

SEQ ID NO: 22 - DNA, 14 bp, synthetic construct
  source 1..14: mol_type = other DNA
catcataggtcatc

SEQ ID NO: 23 - DNA, 14 bp, synthetic construct
  source 1..14: mol_type = other DNA
atcatcataggtcg

SEQ ID NO: 24 - DNA, 19 bp, synthetic construct
  source 1..19: mol_type = other DNA
tcataggtcatcatgctta

SEQ ID NO: 25 - DNA, 19 bp, synthetic construct
  source 1..19: mol_type = other DNA
tcataggtcgtcatgctta

SEQ ID NO: 26 - DNA, 2318 bp, Homo sapiens
  source 1..2318: mol_type = other DNA
gggtttctgcagagtctacttcagaagcggaggcactgggagtccggtttgggattgccaggctgtggttgtgagtctgagcttgtgagcggctgtggcgccccaactcttcgccagcatatcatcccggcaggcgataaactacattcagttgagtctgcaagactgggaggaactggggtgataagaaatctattcactgtcaaggtttattgaagtcaaaatgtccaaaaaaatcagtggcggttctgtggtagagatgcaaggagatgaaatgacacgaatcatttgggaattgattaaagagaaactcatttttccctacgtggaattggatctacatagctatgatttaggcatagagaatcgtgatgccaccaacgaccaagtcaccaaggatgctgcagaagctataaagaagcataatgttggcgtcaaatgtgccactatcactcctgatgagaagagggttgaggagttcaagttgaaacaaatgtggaaatcaccaaatggcaccatacgaaatattctgggtggcacggtcttcagagaagccattatctgcaaaaatatcccccggcttgtgagtggatgggtaaaacctatcatcataggtcgtcatgcttatggggatcaatacagagcaactgattttgttgttcctgggcctggaaaagtagagataacctacacaccaagtgacggaacccaaaaggtgacatacctggtacataactttgaagaaggtggtggtgttgccatggggatgtataatcaagataagtcaattgaagattttgcacacagttccttccaaatggctctgtctaagggttggcctttgtatctgagcaccaaaaacactattctgaagaaatatgatgggcgttttaaagacatctttcaggagatatatgacaagcagtacaagtcccagtttgaagctcaaaagatctggtatgagcataggctcatcgacgacatggtggcccaagctatgaaatcagagggaggcttcatctgggcctgtaaaaactatgatggtgacgtgcagtcggactctgtggcccaagggtatggctctctcggcatgatgaccagcgtgctggtttgtccagatggcaagacagtagaagcagaggctgcccacgggactgtaacccgtcactaccgcatgtaccagaaaggacaggagacgtccaccaatcccattgcttccatttttgcctggaccagagggttagcccacagagcaaagcttgataacaataaagagcttgccttctttgcaaatgctttggaagaagtctctattgagacaattgaggctggcttcatgaccaaggacttggctgcttgcattaaaggtttacccaatgtgcaacgttctgactacttgaatacatttgagttcatggataaacttggagaaaacttgaagatcaaactagctcaggccaaactttaagttcatacctgagctaagaaggataattgtcttttggtaactaggtctacaggtttacatttttctgtgttacactcaaggataaaggcaaaatcaattttgtaatttgtttagaagccagagtttatcttttctataagtttacagcctttttcttatatatacagttattgccacctttgtgaacatggcaagggacttttttacaatttttattttattttctagtaccagcctaggaattcggttagtactcatttgtattcactgtcactttttctcatgttctaattataaatgaccaaaatcaagattgctcaaaagggtaaatgatagccacagtattgctccctaaaatatgcataaagtagaaattcactgccttcccctcctgtccatgaccttgggcacagggaagttctggtgtcatagatatcccgttttgtgaggtagagctgtgcattaaacttgcacatgactggaacgaagtaggagtgcaactcaaatgtgttgaagatactgcagtcatttttgtaaagaccttgctgaatgtttccaatagactaaatactgtttaggccgcaggagagtttggaatccggaataaatactacctggaggtttgtcctctccatttttctctttctcctcctggcctggcctgaatattatactactctaaatagcatatttcatccaagtgcaataatgtaagctgaatcttttttggacttctgctggcctgttttatttcttttatataaatgtgatttctcagaaattgatattaaacactatcttatcttctcctgaactgttgattttaattaaaattaagtgctaattacca